(19) United States
(12) Patent Application Publication
(10) Pub. No.: US 2018/0237515 A1
(43) Pub. Date: Aug. 23, 2018

(54) Compositions And Methods For Treating Cancer

(71) Applicant: MedImmune, LLC, Gaithersburg, MD (US)

(72) Inventors: Lilian Emilia VAN VLERKEN-YSLA, Gaithersburg, MD (US); Elaine Marie HURT, Gaithersburg, MD (US)

(21) Appl. No.: 15/743,384

(22) PCT Filed: Jul. 13, 2016
(86) PCT No.: PCT/US16/42100

§ 371 Date: Jan. 10, 2018

Related U.S. Application Data
(60) Provisional application No. 62/192,204, filed on Jul. 14, 2015.

(51) Int. Cl. C07K 16/24; G01N 33/574; A61P 35/00

(52) U.S. Cl. CPC C07K 16/24; G01N 33/574; A61K 2039/505; C07K 2317/76; A61P 35/00

(57) ABSTRACT

The disclosure generally provides compositions and methods that are useful in the treatment of cancer. More specifically, the methods and compositions may be used to detect, quantify, inhibit, kill, differentiate, or eliminate cancer stem cells (CSCs) and may be used in the treatment of cancers associated with CSCs, and particularly cancers and CSCs that express CCL20 and/or CCR6.

### REFERENCE TO SEQUENCE LISTING SUBMITTED ELECTRONICLY

This application incorporates by reference a Sequence Listing submitted with this application in computer readable form (CRF) as a text file entitled “CCL20-100P1_SeqList_ST25” created on Jul. 12, 2016, and having a size of 19.2 KB.

### BACKGROUND

Cancer stem cells (CSCs), as their name implies, are a subset of cancer cells with stem cell like characteristics of pluripotency and unlimited self-renewal. As such, it is believed that this subpopulation of cells is responsible for tumor formation and adaptation to its environment. Several lines of evidence now indicate that CSCs are likely responsible for drug resistance, metastasis, and relapse of cancer, particularly in instances with minimal residual disease. In fact, recent clinical evidence showed that the fraction of breast cancer cells that survived following standard-of-care therapy was enriched in cells bearing a CSC signature (Creighton et al., 2009). In a similar study, patients with 5q deletion MDS were also found to have residual populations of malignant stem cells in their bone marrow, despite having entered clinical and cytogenetic remission (Tehranchi et al., 2010). In many clinical indications, it is now believed that despite initial therapeutic response, the retention of a distinct subset of resistant cancer cells can lead to relapse and potentially metastasis.

Since their initial identification, cancer stem cells have been discovered and validated in many tumor types. Following the original identification of a CSC in a model of AML (Lapidot et al., 1994), CSCs were discovered and validated in a number of hematological and solid tumor malignancies. The first evidence of CSCs in solid tumors came from breast cancer, where CSCs were found to bear a CD44⁺/CD24⁻ surface marker phenotype (Al-Hajj et al., 2003). CSCs in pancreatic cancer have also been well characterized. While various reports identify pancreatic CSCs as either Epcam⁺/CD44⁺/CD24⁺ (Li et al., 2007) or CD133⁺ (Hermann et al., 2007), in the inventors' hands, the triple positive Epcam⁺/CD44⁺/CD24⁺ signature tracks best with the tumorigenic phenotype of a CSC (van Vlerken et al., 2013). A number of reports identify CSCs in colorectal cancer, and research has identified a stem cell origin in the mouse for intestinal and colorectal tumors (Barker et al., 2009). Nevertheless, the state of the art in this field has not reached a consensus regarding the surface marker phenotype for human colorectal CSCs. While many surface markers have been reported, such as CD66 (Gemei et al., 2013), LGR5 (Hirsch et al., 2014), and CD44 and CD133 (Wang et al., 2012), the field lacks any robust confirmation that any of these surface marker phenotypes may represent a consensus for human colorectal CSCs, allowing for other techniques such as non-adherent sphere growth (e.g., colospheres for colorectal CSCs) to find common use in identifying the presence of CSCs.

Drugs that target CSCs are emerging as a critical component of any successful therapy against cancer. Current research is showing that CSCs are responsible for chemoresistance, metastasis, and ultimately relapse of the cancer, even after an initial successful therapeutic intervention. While CSCs are driven by the same major self-renewal pathways that drive pluripotency of embryonic stem cells, knowledge of other cellular pathways that drive CSC activity is still limited.

The availability of additional compositions and methods for treatment of cancers, including methods for reducing tumorigenicity of cancer and inhibiting or killing cancer cells, such as cancer stem cells (e.g., inducing apoptosis or differentiation), would allow for more therapeutic options and hold the potential for better clinical outcomes such as disease remission and/or improvement of patient quality of life.

### SUMMARY OF THE DISCLOSURE

In one aspect, the disclosure relates to a method for inhibiting proliferation of a cancer stem cell (CSC) comprising contacting the CSC with an inhibitor of CCL20 and/or an inhibitor of CCR6 in an amount effective to inhibit proliferation and/or reduce survival of the CSC.

In an aspect, the disclosure relates to a method for treating a therapeutically-resistant cancer in a subject who has previously received a therapy and who is in need of treatment, comprising administering to the subject an inhibitor of CCL20 and/or an inhibitor of CCR6 in an amount effective to inhibit or kill cancer stem cells (CSCs) present in the therapeutically-resistant cancer. In some embodiments, the method may comprise administering to a subject an inhibitor of CCL20 and/or an inhibitor of CCR6 in an amount effective to induce differentiation of cancer stem cells (CSCs) present in the therapeutically-resistant cancer.

In an aspect, the disclosure relates to a method for treating a therapeutically-resistant cancer in a subject who has previously received a therapy and who is in need of treatment, comprising administering to the subject an inhibitor of CCL20 and/or an inhibitor of CCR6 in an amount effective to treat the therapeutically-resistant cancer.

In an aspect, the disclosure relates to a method for treating a cancer that comprises cancer stem cells (CSCs), the method comprising administering to a subject in need of treatment an inhibitor of CCL20 and/or an inhibitor of CCR6 in an amount effective to target and inhibit or kill the CSCs in the cancer. In some embodiments, the method may comprise administering to a subject an inhibitor of CCL20 and/or an inhibitor of CCR6 in an amount effective to induce differentiation of cancer stem cells (CSCs) present in the cancer.

In an aspect, the disclosure relates to a method for treating a cancer that comprises cancer stem cells (CSCs), the method comprising administering to a subject in need of treatment an inhibitor of CCL20 and/or an inhibitor of CCR6 in an amount effective to treat the cancer.

In an aspect, the disclosure relates to a method for treating cancer in a subject who has recurring or relapsed cancer comprising administering to a subject an inhibitor of CCL20 and/or an inhibitor of CCR6 in an amount effective to inhibit or kill CSCs in the cancer. In some embodiments, the method may comprise administering to the subject an inhibitor of CCL20 and/or an inhibitor of CCR6 in an amount effective to induce differentiation of cancer stem cells (CSCs) present in the recurring or relapsed cancer.

In an aspect, the disclosure relates to a method for treating cancer in a subject who has recurring or relapsed cancer comprising administering to a subject an inhibitor of CCL20 and/or an inhibitor of CCR6 in an amount effective to treat the cancer.

In one aspect, the disclosure relates to a method for preventing a recurrence or a relapse of cancer, comprising administering to a subject in need of prevention of recurrence or relapse of cancer an inhibitor of CCL20 and/or an inhibitor of CCR6 in an amount effective to prevent recurrence or relapse of cancer.

In a further aspect, the disclosure relates to a method for reducing the risk of cancer relapse in a subject who has cancer comprising administering to the subject an inhibitor of CCL20 and/or an inhibitor of CCR6 in an amount effective to inhibit or kill CSCs in the cancer, thereby reducing the risk of cancer relapse in the subject. In some embodiments, the method may comprise administering to the subject an inhibitor of CCL20 and/or an inhibitor of CCR6 in an amount effective to induce differentiation of cancer stem cells (CSCs) present in the cancer and reduce the risk of relapse or recurrence of the cancer.

In another aspect, the disclosure relates to a method of eliminating a cancer stem cell (CSC) comprising contacting the CSC with an inhibitor of CCL20 and/or an inhibitor of CCR6 in an amount effective to eliminate the CSC.

In an aspect, the disclosure relates to a method of selectively reducing the number of cancer stem cells (CSCs) in a population of cancer cells comprising contacting the population of cancer cells with an inhibitor of CCL20 and/or an inhibitor of CCR6 in an amount effective to inhibit or kill CSCs in the population of cancer cells. In some embodiments, the method may comprise administering to the subject an inhibitor of CCL20 and/or an inhibitor of CCR6 in an amount effective to induce differentiation of cancer stem cells (CSCs) in the population of cancer cells and reduce the number of CSCs.

In a further aspect, the disclosure provides a method for reducing or inhibiting tumor growth comprising contacting the tumor with an inhibitor of CCL20 and/or an inhibitor of CCR6 in an amount effective to reduce or inhibit tumor growth. In a related aspect, the disclosure provides a method for reducing or inhibiting tumor growth in a patient comprising administering to the patient an inhibitor of CCL20 and/or an inhibitor of CCR6 in an amount effective to reduce or inhibit tumor growth.

In another aspect, the disclosure provides a method for reducing the size of a tumor comprising contacting the tumor with an inhibitor of CCL20 and/or an inhibitor of CCR6 in an amount effective to reduce tumor size. In a related aspect, the disclosure relates to a method for reducing the size of a tumor in a patient comprising administering to the patient an inhibitor of CCL20 and/or an inhibitor of CCR6 in an amount effective to reduce tumor size.

In another aspect, the disclosure provides a method for inhibiting, reducing, or preventing tumor invasiveness or metastasis comprising contacting the tumor with an inhibitor of CCL20 and/or an inhibitor of CCR6 in an amount effective to inhibit, reduce, or prevent tumor invasiveness or metastasis. In a related aspect, the disclosure relates to a method for inhibiting, reducing, or preventing tumor invasiveness or metastasis in a patient comprising administering to the patient an inhibitor of CCL20 and/or an inhibitor of CCR6 in an amount effective to inhibit, reduce, or prevent tumor invasiveness or metastasis.

The disclosure also relates to one or more uses of an inhibitor of CCL20 and/or an inhibitor of CCR6, or a combination thereof in a composition, including therapeutic and pharmaceutical compositions. In embodiments, the uses and compositions comprise the inhibitor(s) in amounts effective to provide for the various methods disclosed herein. In embodiments, the one or more uses relate to uses for the manufacture of a medicament for treating (e.g., killing, eliminating, inhibiting, reducing the progression and/or rate of progression of cell proliferation, reducing the progression and/or rate of progression of a disease state, reducing the ability for self-renewal and expansion (e.g., inducing differentiation)) one or more of the tumors, cancer stem cells, and/or cancers described herein.

In another aspect, the disclosure provides methods for diagnosis, prognosis, quantification, identification, and/or detection of the presence of a cancer stem cell (CSC) in a sample comprising cancer cells, wherein the method comprises:
contacting the sample with an agent that binds to a CCL20 nucleic acid sequence or a CCL20 amino acid sequence;
detecting the presence or absence of binding between the agent and the CCL20 nucleic acid sequence or the CCL20 amino acid sequence; and
identifying the presence of the CSC in the sample upon detection of binding between the agent and the CCL20 nucleic acid sequence or the CCL20 amino acid sequence.

In embodiments of this aspect, the method can further comprise one or more of:
quantifying the amount of the CCL20 nucleic acid sequence or the CCL20 amino acid sequence in the sample;
comparing the amount of the CCL20 nucleic acid sequence or the CCL20 amino acid sequence to a reference level of CCL20 nucleic acid or CCL20 amino acid; and/or
identifying the presence of the CSC in the sample when the detected amount is greater than the reference level.

In yet further embodiments, the method may comprise an agent comprising a detectable moiety. In some embodiments, the agent may comprise a nucleic acid sequence that hybridizes to at least a portion of the CCL20 nucleic acid sequence under stringent hybridization conditions. In some embodiments, the agent may comprise an antibody that specifically binds to at least a portion of the CCL20 amino acid sequence.

In various embodiments of the above aspects, the methods relate to a tumor and/or a CSC, or a population of CSCs expressing CCL20. In some embodiments of the above aspects, the methods relate to a tumor and/or a CSC, or a population of CSCs expressing CCR6. In further embodiments, the tumor and/or a CSC, or a population of CSCs may comprise sphere-forming CSCs.

In various embodiments of the above aspects, the methods relate to a tumor and/or a cancer selected from colorectal cancer, pancreatic cancer, lung cancer, head and neck cancer, gastric cancer, hepatocellular carcinoma, and breast cancer.

The aspects and embodiments described herein relate to methods that comprise an inhibitor of CCL20, an inhibitor of CCR6, or a combination of inhibitors of CCL20 and/or CCR6. In some embodiments the inhibitor comprises an antibody that binds CCL20, an antibody that binds CCR6, a small molecule inhibitor of CCL20, a small molecule inhibitor of CCR6, a siRNA that hybridizes to a nucleic acid encoding CCL20, or a siRNA that hybridizes to a nucleic acid encoding CCR6, or any combinations thereof.

The above aspects and embodiment also relate to methods, uses, and compositions that further comprise an additional therapy for treatment of diseases relating to angiogenesis, tumorigenesis, and cancers and can include a therapeutic regimen such as, for example, a chemotherapy, a radiotherapy, an immunotherapy, or any other active agent or therapy that is known in the art.

Other aspects will be apparent to one of skill in the art upon review of the description and exemplary depictions that follow.

### BRIEF DESCRIPTION OF THE DRAWINGS

For the purpose of illustrating the disclosure, there are depicted in the drawings certain aspects of the disclosure. However, the disclosure is not limited to the precise arrangements and instrumentalities of the aspects depicted in the drawings.

FIG. 1A-1E shows that CSCs are found to have increased expression of CCL20 over non-CSCs. CSC-enriching sphere culture promotes the secretion of CCL20 compared to standard monolayer culture in 4 out of 8 breast cancer lines (A), 2 out of 4 pancreatic cancer lines (B), and 6 out of 7 colorectal cancer lines (C) tested. CCL20 levels were measured in picogram/milliliter (pg/mL) from conditioned medium of cells after 4 days in culture by ELISA and pg/mL CCL20 levels were subsequently converted to fold-change compared to monolayer cultured cells of the same line. FACS isolated CD44^{high/+} CD24^{low/-} CSCs from 2 breast cell lines (MDAMB468 and HCC1937) have elevated CCL20 mRNA levels compared with CD44^{low/-} CD24^{low/-} non-CSCs isolated from the same lines (D). Similarly, isolated CSCs from 1 breast (BR_PDX_1) and 2 pancreatic (PA_PDX_1 and PA_PDX_2) patient-derived xenograft (PDX) models also reveals elevated CCL20 mRNA levels compared with non-CSCs isolated from the same line (E). CSCs for breast PDX models were identified as Epcam⁺ CD44⁺ CD24⁻, and for pancreatic PDX models as Epcam⁺ CD44⁺ CD24⁺, while PDX breast non-CSCs were Epcam⁺ CD44⁻ CD24⁻, and pancreatic non-CSCs were Epcam⁻ CD44⁻ CD24⁻ For panels A-C, n=3 biological replicates/condition/cell line; ***, **, and * denote statistical significance of p<0.001, p<0.01, and p<0.05 respectively between spheres and monolayer culture as determined by t-test. For panels D and E, n=2 technical replicates/condition/cell line. RE=relative expression of Ct values normalized to 18S ribosomal RNA and expressed compared to non-CSC levels.

FIG. 2 shows CCL20 secretion in sphere culture. Average±SEM for CCL20 levels measured in conditioned media of sphere cultured cells harvested at day 4. Absolute CCL20 levels were measured by ELISA relative to a standard curve. N=3 biological replicates/cell line.

FIG. 3A-3F shows that CSCs directly co-localize with a CCL20+ phenotype. CSCs from monolayer culture of the Bxpc3 pancreatic model are identified as Epcam⁺ CD44^{high} CD24^{high} by flow cytometry (A), while breast CSCs from the monolayer HCC1937 model are identified as CD44^{high} CD24⁻ (D). In both Bxpc3 (B) and HCC1937 (E), CSCs are found to have 1.5-fold greater CCL20 expression than the average of the total tumor cell population (total tumor) does as determined by median fluorescence intensity (Median: Comp-APC A) of APC-tagged anti-CCL20. Furthermore, in both Bxpc3 (C) and HCC1937 (F), CSCs, illustrated as black dots, appear to predominantly overlap with a CCL20⁺ CCR6⁺ population of cells as determined by multicolor flow cytometry. Scatter of the total tumor cells is illustrated as grey dots for comparison.

FIG. 4A-4F illustrates CSC-enriched sphere culture causes a dramatic expansion of the CCL20⁺CCR6⁺ population. The frequency of CCL20⁺CCR6⁺ dramatically increases in the Bxpc3 (A, B) and Panc1 (C, D) pancreatic cancer models when cells are cultured under sphere forming conditions (B, D) compared with monolayer culture (A, C) as determined by multicolor flow cytometry. Furthermore, pancreatic CSCs identified from sphere culture as Epcam⁺CD44⁺CD24⁺, illustrated as black dots, overlap predominantly with the CCL20⁺CCR6⁺ population in Bxpc3 (E) and Panc1 (F) models. Scatter of the total tumor cells is illustrated as grey dots for comparison.

FIG. 5A-5D shows that addition of purified CCL20 to culture boosts the growth of CSC-enriched spheres, but not standard monolayer cultured cells. Bxpc3 pancreatic cancer (A), Aspc1 pancreatic cancer (B), and SUM159 breast cancer (C) models were stimulated with recombinant human CCL20 (rhCCL20) at 0, 25, 50, 100, and 200 pg/mL for 4 days in either sphere or monolayer culture, whereby CCL20 was dosed at the time of plating. BT549 cells (D) illustrate that the potentiation of CCL20 on sphere growth is seen with CCL20 from 2 separate commercial sources, R&D systems (CCL20 A) and Life Technologies (CCL20 B), both dosed at 50 pg/mL. The specificity of CCL20 in mediating this growth stimulation was confirmed when addition of an anti-CCL20 monoclonal antibody at 15 μg/mL nullified this effect for both CCL20 A and CCL20 B (D). n=5 biological replicates/treatment/cell line. ***, **, and * denote statistical significance of p<0.001, p<0.01, and p<0.05 respectively between spheres and monolayer culture at matched concentrations of CCL20 as determined by t-test. ## and # denotes statistical significance of p<0.01 and p<0.05 respectively between basal and CCL20 stimulated spheres, and ̂ denotes statistical significance of p<0.05 between CCL20 stimulated cells with and without addition of anti-CCL20, as determined by 1-way ANOVA with Tukey's multiple comparisons.

FIG. 6A-6D shows that stimulation of cancer cells with CCL20 directly boosts the frequency of CSCs in culture. Addition of 200 pg/mL recombinant human CCL20 to cells in sphere culture directly increases the percentage of CSCs after 4 days in breast and pancreatic cancer models, seen in (A) MDAMB468 breast cancer cells characterized as CD44⁺ CD24⁻, (B) in Bxpc3 pancreatic cancer and (C) in Aspc1 pancreatic cancer cells, both characterized as Epcam^{high} CD44^{high} CD24^{high}. Bxpc3 spheres stimulated for 4 days with 200 pg/mL show a corresponding increase in the stemness genes, NANOG, SOX2, OCT3/4, BMI1, and EZH2 (D). Panels A-C, n=3 biological replicates/treatment/cell line. * denotes statistical significance of p<0.05 between cells treated with or without CCL20 as determined by one-sided t-test. Panel D, n=2 technical replicates/treatment; RE=relative expression of Ct values normalized to 18S ribosomal RNA and expressed compared to −CCL20 levels.

FIG. 7A-7C shows stimulation of spheres with CCL20 induces the expression of stemness genes. Relative expression (RE) of stemness genes NANOG, SOX2, OCT3/4, EZH2, AND BMI1 in Hs578T (A), Panc1 (B), and SUM159 (C) spheres treated for 4 days with 100 pg/mL recombinant human CCL20 (+ CCL20) compared with untreated spheres (− CCL20). RE=relative expression of Ct values normalized to 18S ribosomal RNA and expressed compared to −CCL20 levels. N=2 technical replicates/treatment/cell line.

FIG. 8A-8D shows CCL20 regulates CSCs by signaling through its receptor CCR6. (A) Recombinant human CCL20 (rhCCL20) loses the ability to stimulate sphere growth by 4 days after treatment initiation in Lovo colorectal cancer cells following knockdown of CCR6 with 20 nM siRNA, while cells transfected with non-target control siRNA (20 nM) retained their growth response to CCL20. (B) CCR6⁺ cells show an increase in stemness genes 48 hours after stimulation with CCL20 (100 pg/mL) compared with CCR6⁻ cells isolated from the NCIH508 colorectal cancer model. (C-D) Inhibition of the CCL20-CCR6 axis, either by knockdown of CCL20 or CCR6 (20 nM siRNA) resulted in a similar level of sphere growth inhibition of NCIH508 colorectal cancer cells (C) and Hs578T breast cancer cells (D) compared with control siRNA (20 nM) treatment. Panel A, n=5 biological replicates/condition. ***, **, and * denote statistical significance of p<0.001, p<0.01, and p<0.05 respectively between control siRNA and CCR6 siRNA transfected cells at matched concentrations of CCL20 as determined by t-test. Panel B, RE=relative expression of Ct values normalized to 18S ribosomal RNA and expressed compared to CCR6⁻ levels. Panel C, n=5 biological replicates/treatment. *** denotes statistical significance of p<0.001 from control siRNA, as determined by 1-way ANOVA with Tukey's multiple comparisons.

FIG. 9A-9E shows treatment with a neutralizing monoclonal CCL20 antibody significantly reduces CSCs measured by sphere growth and flow cytometry. Sphere growth is reduced following a 4 day treatment with 10 μg/mL CCL20 IgG, compared with 10 μg/mL isotype control IgG treatment in (A) Hs578T breast cancer cells, (B) NCIH508 colorectal cancer cells, and (C) Panc1 pancreatic cancer cells. A single dose 10 μg/mL CCL20 IgG treatment reduces the percentage of Epcam⁺CD44⁺CD24⁺ pancreatic CSCs in Bxpc3 (D) and Panc1 (E) spheres compared with isotype control IgG treatment at the same 10 μg/mL dose. Panels A-C, n=5 biological replicates/treatment/cell line. Panels D-E, n=3 biological replicates/treatment/cell line. ***, **, and * denotes statistical significance of p<0.001, p<0.01, and p<0.05 between control IgG and CCL20 IgG treated cells, respectively, as determined by t-test.

FIG. 10 shows treatment with a neutralizing monoclonal CCL20 antibody significantly inhibits chemotactic migration of CD4+ T cells towards CCL20. CD4+ T cells were isolated from human healthy donor blood and allowed to migrate towards 10 nM recombinant human CCL20 with 30 nM isotype IgG or anti-CCL20 IgG in transwell migration plates with a polycarbonate filter and 5 μM pores for 3 hours. n=3 biological replicates/treatment/donor. ** and *** denotes statistical significance of p<0.01 and p<0.001 between isotype IgG and CCL20 IgG, respectively, as determined by t-test.

FIG. 11A-11I shows a multiparameter flow cytometric analysis of TR1 phenotype. 57% of live cells in the population stained positive for CCR6 (A). Of these CCR6+ cells, 93% were found to be CD3+/CD4+ (B). Of the CD3+/CD4+ cells, 94% were found to be CD25+ (C), and of these CD25+ cells, 85% were confirmed to be FOXP3+ (D). Positive vs. negative gating was determined by full-minus-one (FMO−) staining whereby samples contained all the stains in the panel minus CD3 (E), CD4 (F), CD25 (G), FoxP3 (H), or CCR6 (I).

FIG. 12A-12C shows that TR1 cells migrate towards CCL20 secreted by CSCs. Chemotactic attraction of TR1 cells towards 100 nM CCL20 was determined by a transwell migration assay compared with non-specific migration (no CCL20) and compared to inhibition of migration through the addition of neutralizing anti-CCL20 IgG (667 nM) (A). CCL20 concentration in CSC conditioned medium (CSC CM), generated from sphere culture of NCIH508 cells, was determined by ELISA. CCL20 levels in unused SCM media is plotted by comparison to ensure that CCL20 was produced from NCIH508 CSCs (B). Chemotactic attraction of TR1 cells towards CSC CM was also determined by a transwell migration assay compared with non-specific migration (no CCL20) and compared to inhibition of migration through the addition of neutralizing anti-CCL20 IgG (667 nM) or isotype control IgG (667 nM). n=4 replicates/treatment.

FIG. 13A-13E shows gating controls for Bxpc3 and HCC1937 flow cytometry data from FIG. 3. Full-minus-one (FMO) stains were used to define positive vs. negative staining gates for each protein in multicolor flow cytometry experiments. Bxpc3 cells (top row, A-C) were stained with a 5-color panel for Epcam, CD24, CD44, CCL20, and CCR6, while HCC1937 cells (bottom row, D-E) were stained with a 4 color panel for all the above except Epcam. (A) Full-minus-Epcam (FMO−Epcam) (dashed line, grey shading) compared with the full-stained sample (solid line, white shading). (B, D) Full-minus-CD44 (FMO−CD44) is depicted in grey and full-minus-CD24 (FMO−CD24) is depicted in green. (C, E) Full-minus-CCL20 (FMO−CCL20) is depicted in black and full-minus-CCR6 (FMO−CCR6) is depicted in grey.

FIG. 14A-14B shows gating controls for Bxpc3 and Panc1 flow cytometry data from FIG. 4. Monolayer and sphere cultured Bxpc3 and Panc1 cells were stained with a 5-color panel for Epcam, CD24, CD44, CCL20, and CCR6. For each experiment, gating controls were run on monolayer cultured cells. Full-minus-CCL20 (FMO−CCL20) is depicted in grey while full-minus-CCR6 (FMO−CCR6) is depicted in black, for both Bxpc3 (A) and Panc1 (B) monolayer cultured cells.

FIG. 15A-15D shows gating controls for Bxpc3 and Panc1 flow cytometry data from FIGS. 9 D, E. Sphere cultured Bxpc3 (A, B) and Panc1 (C, D) cells were stained with a 5-color panel for Epcam, CD24, CD44, CCL20, and CCR6. For each experiment, gating controls were run on monolayer cultured cells. (A, C) Full-minus-Epcam (FMO−Epcam) (dashed line, grey shading) compared with the full-stained sample (solid line, white shading). (B, D) Full-minus-CD24 (FMO−CD24) is depicted in black while full-minus-CD44 (FMO−CD44) is depicted in grey.

FIG. 16A-16B depicts how CCL20 IgG neutralizes CCL20-stimulated CCR6 pathway activity measured through (A) cAMP and (B) beta-arrestin activity. cAMP assay was performed using Ad293 cells overexpressing human CCR6, stimulated with 1 nM CCL20 and 4 mM forskolin, while the beta-arrestin assay was performed using CHOKI cells overexpressing human CCR6, stimulated with 6 nM CCL20. n=2 technical replicates/dose/as say.

FIG. 17A-17B shows (A) Take rate of Panc1 tumors (% tumor-bearing animals) is reduced when Panc1 spheres are pre-treated with 10 ug/mL CCL20 IgG compared to Panc1 spheres that were pre-treated with 10 ug/mL isotype IgG for 4 days in-vitro prior to tumor inoculation, whereby tumor take is measured from the first instance of palpable tumor formation. (B) Pre-treating Panc1 spheres with 10 ug/mL CCL20 IgG prior to inoculation leads to significantly reduced tumor growth compared with isotype IgG pre-treated Panc1 spheres (10 ug/mL). For panel A, median time to tumor take was determined by Kaplan-Meier survival analysis. For panel B, **, and * denotes statistical significance of p<0.01 and p<0.05 between isotype IgG and CCL20 IgG treated tumors, respectively, as determined by 2-way ANOVA.

FIG. 18 shows anti-mouse CCL20 IgG (clone 114908) neutralizes CCL20-stimulated CCR6 pathway activity measured through beta-arrestin activity. The assay was performed using CHOKI cells overexpressing mouse CCR6, stimulated with 1.2 nM mouse CCL20.

FIG. 19 shows a representative flow cytometry diagram depicting gating and immune cell analysis in the spleen. Cells were first gated on cd45 as a pan-leukocyte marker. Next T cells were identified by cd3 staining, dendritic cells by cd11c for total cells or cd11c/cd83 to separate mature and immature dendritic cells, and neutrophils/granulocyte myeloid derived suppressive cells (gMDSC) by cd11b/ly6g. Cd3+ T cells were further subsetted for cd4 and cd8 staining, and cd4+ cells were then separated into foxp3+ T_{regs} or rorgt+ Th₁₇ cells. All gates were determined by FMO controls and kept constant across all samples.

FIG. 20 shows a representative flow cytometry diagram depicting gating and immune cell analysis in the tumor. Cells were first gated on cd45 as a pan-leukocyte marker. Next T cells were identified by cd3 staining, dendritic cells by cd11c for total cells or cd11c/cd83 to separate mature and immature dendritic cells, and neutrophils/granulocyte myeloid derived suppressive cells (gMDSC) by cd11b/ly6g. Cd3+ T cells were further subsetted for cd4 and cd8 staining, and cd4+ cells were then separated into foxp3+ T_{regs} or rorgt+ Th₁₇ cells. All gates were determined by FMO controls and kept constant across all samples.

FIG. 21A-21E shows the frequency of immune cell populations in the spleens of 4T1 tumor-bearing mice dosed with 10 mg per kg anti-mouse CCL20 IgG or isotype control IgG, twice weekly for 4 doses. (A) cd3+ cd4+ foxp3+ T_{reg} cells, (B) cd3+ cd4+ rorγt+ Th₁₇ cells, (C) cd11b+ total dendritic cells, (D) cd11b+ cd83− immature dendritic cells, and (E) cd11b+ cd83+ mature dendritic cells. All cell frequencies are depicted as % of total leukocytes (cd45+ cells). n=5 animals/group. Statistical significance was determined by two-sided student's t-test.

FIG. 22A-22E shows the frequency of immune cell populations in the tumors of 4T1 tumor-bearing mice dosed with 10 mg per kg anti-mouse CCL20 IgG or isotype control IgG, twice weekly for 4 doses. (A) cd3+ cd4+ foxp3+ T_{reg} cells, (B) cd3+ cd4+ rorγt+ Th₁₇ cells, (C) cd11b+ total dendritic cells, (D) cd11b+ cd83− immature dendritic cells, and (E) cd11b+ cd83+ mature dendritic cells. All cell frequencies are depicted as % of total leukocytes (cd45+ cells). n=5 animals/group. Statistical significance was determined by two-sided student's t-test.

### DETAILED DESCRIPTION

As described herein, the inventors provide description that demonstrates, for the first time, that CCL20 is overexpressed by and is functionally relevant for CSCs. While reports have referred to potential CCL20 expression in some cancers, no reports have identified and related CCL20 expression and function to any CSC phenotype, which provides for new and unexpected methods for treating cancer and inhibiting CSCs. As illustrated in certain aspects and non-limiting embodiments disclosed herein, the novel methods that target and inhibit CCL20 activity are useful and effective against cancers that comprise and exhibit cells having a CSC phenotype. In some illustrative embodiments, the methods and agents disclosed herein may be used to target cancers comprising CSCs that express CCL20 and/or CCR6 including, for example, pancreatic cancer, colorectal cancer, hepatocellular carcinoma, gastric cancer, lung cancer, head and neck cancer, and breast cancer. Thus, the disclosure provides for novel targeting strategies that include methods and agents that inhibit CSCs and can be used to inhibit and/or treat various cancers in patients suffering from cancer and improve quality of life.

The disclosure includes a description and overview of whole genome mRNA analysis on breast CSCs which surprisingly identified that changes in the expression of the gene CCL20 appeared to correlate directly with enrichment or inhibition of CSCs (van Vlerken et al., 2013). Chemokine (C-C motif) ligand 20 (CCL20) is also commonly referred to as macrophage inflammatory protein 3-alpha (MIP-3a) or liver activation regulated chemokine (LARC) (Schutyser et al., 2003). CCL20 functions normally as a chemotactic factor for the recruitment of T-, B-, and immature dendritic-cells, and is produced predominantly by cells of the liver, lung, and gastrointestinal tract (Schutyser et al., 2003). Chemokine receptor 6 (CCR6) has been identified as the receptor for CCL20 (Baba et al., 1997) and, to date, is still the lone functional receptor identified for the CCL20 ligand. Despite its role in immune cell recruitment, several lines of evidence have hinted at an association for CCL20 in cancer. One report identified CCL20 as a factor contributing to pathogenesis of pancreatic cancer, whereby the authors concluded a direct effect for CCL20 in stimulating pancreatic cancer growth (Kleeff et al., 1999). While the early identification was associated with pancreatic cancer, the largest body of evidence to date links CCL20 expression with colorectal cancer (Ghadjar et al., 2009). In fact, recent work proposed high serum levels of CCL20 as a biomarker for poor prognosis in colorectal cancer (Iwata et al., 2013). The inventors have identified CCL20 in breast cancer models which has been corroborated by external evidence that shows CCL20 can directly affect migration and proliferation of breast cancer cells (Marsigliante et al., 2013). Although some reports suggest a direct autocrine effect of the CCL20-CCR6 axis on tumor cells, the majority of reports propose that CCL20 overexpression by tumor cells serves to promote immune cell recruitment. While the current disclosure implicates an autocrine function for CCL20 on CSCs, the disclosure also provides that CCL20 secretion by CSCs can aide in recruitment of tumor immune infiltrate. For example, evidence suggests that Tregs (Chen et al. 2011, Liu et al. 2011, Zhang et al. 2014) and Th17 cells (Yu et al. 2015) in particular would be prone to tumor infiltration in response to CCL20 production, in addition to B-cells and dendritic cells that are also likely mobilized towards CCL20 produced by CSCs.

As disclosed herein, the inventors uncover a role for chemokine (C-C motif) ligand 20 (CCL20) in CSC growth and activity, a finding that is entirely novel to CSC biology. The disclosure provides illustrative embodiments demonstrating that CSCs of representative breast, pancreatic, and colorectal cancer models overexpress CCL20, and that in turn CCL20 can drive CSC self-renewal and proliferation by signaling through its receptor CCR6, also expressed on CSCs. Neutralizing CCL20 with a monoclonal antibody has a significant effect on CSC inhibition, supporting the great potential for therapies targeting this chemokine to reduce CSCs in the tumor.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

The terms “about” and “approximately” in the context of the present invention denote an interval of accuracy that a person skilled in the art will understand to still ensure the technical effect of the feature in question. The term typically encompasses a deviation from the indicated numerical value of about +/−10%, or about +/−5%.

The determination of percent identity between two sequences is preferably accomplished using the mathematical algorithm of Karlin and Altschul (1993) Proc. Natl. Acad. Sci USA 90: 5873-5877. Such an algorithm is e.g. incorporated into the BLASTn and BLASTp programs of Altschul et al. (1990) J. Mol. Biol. 215: 403-410 available at the NCBI website. The determination of percent identity is preferably performed with the standard parameters of the BLASTn and BLASTp programs.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., multispecifc antibodies, e.g., PCT publication WO2009018386, PCT Application No. PCT/US 2012/045229, incorporated herein by reference in its entirety), biMabs, human antibodies, humanized antibodies, camelised antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain at least one antigen-binding site. Antibodies also include peptide fusions with antibodies or portions thereof such as a protein fused to an Fc domain. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens).

The terms “chemokine receptor CCR6” or “CCR6 receptor”, or “CCR6” and “Chemokine ligand 20” or “CCL20” as used herein refer to biological molecules (nucleic acid molecules and amino acid molecules) that are known in the art. One of ordinary skill in the art will be able to identify the polynucleotide and amino acid sequences of CCR6 receptor and CCL20, as well as any orthologous and splice variant isoforms of CCR6 and CCL20 from any sequence database (e.g. the NCBI database).

In some embodiments the terms “CCR6 receptor” or “CCL20” refer to a mammalian CCR6 receptor and CCL20, and in certain embodiments, refer to human CCR6 receptor and human CCL20. As noted above, the sequences of human CCR6 receptor are available from public sequence databases such as, for example, the NCBI database (accession number NM_004367.5 (transcript variant 1; SEQ ID NO:1, encoding SEQ ID NO:2) or NM_031409.3 (transcript variant 2; SEQ ID NO: 3, encoding SEQ ID NO:4). Similarly, the sequences of human CCL20 are publicly available (NCBI accession number NM_004591.2 (transcript variant 1; SEQ ID NO:5, encoding SEQ ID NO:6) or NM_001130046.1 (transcript variant 2; SEQ ID NO:7, encoding SEQ ID NO:8). Other orthologs and variants will be known and/or identifiable to one of skill in the art.

CCR6 is sometimes also referred to as CD196 or CD196 antigen. A number of other terms for CCR6 have been used in the art with varying frequency including, for example, CC-CKR-6, C-C-CKR-6, Chemokine (C-C Motif) Receptor 6, Chemokine (C-C) Receptor 6, C-C Chemokine Receptor Type 6, CKRL3, CKR-L3, Chemokine Receptor-Like 3, STRL22, CMKBR6, G Protein-Coupled Receptor 29, GPR29, Seven-Transmembrane Receptor, Lymphocyte 22, GPRCY4, GPR-CY4, DRY6, LARC Receptor, and BN-1.

Similarly, CCL20, or C-C Motif Chemokine 20, is also known as macrophage inflammatory protein-3α (MIP3 α, MIP3-alpha, MIP3A, MIP3a, MIP-3a), liver and activation-regulated chemokine (LARC), CC Chemokine LARC, SCYA20, Small-inducible cytokine A20, Small Inducible Cytokine Subfamily A (Cys-Cys), Member 20, ST38, CKb4, Beta Chemokine Exodus-1, Beta-Chemokine Exodus-1, or exodus-1. As noted above, CCL20 is a 9 kDa CC-type chemokine, which is expressed constitutively at low levels by keratinocytes in various tissues (e.g., skin, intestinal mucosa, liver). While there is redundancy in human chemokine network, CCL20 is the unique chemokine ligand of its receptor, CCR6. CCL20 expression has been described in a variety of human neoplasms, including colorectal, lung, pancreatic and breast human adenocarcinomas, malignant glioma, leukemia, lymphoma and melanoma. To date and prior to the instant disclosure, however, the in vivo role of CCL20, in particular in the context of cancer and cancer therapy, has not been established.

### A. Methods

The disclosure provides a number of aspects and embodiments relating to methods that incorporate an inhibitor of CCL20 or an inhibitor of CCR6, or combinations thereof. As discussed herein, these aspects and embodiments encompass various methods relating to disease, including methods of treatment, methods of prevention, methods of inhibition and/or slowing progression, methods of reducing risk of relapse and/or recurrence of disease; methods of targeting and/or killing particular cell populations; methods of inducing differentiation and/or reducing the self-renewal and expansion capacity of CSCs; methods of detecting, diagnosing, and/or quantifying particular cell populations and/or diseases, including determining disease stage, and methods of disease monitoring and/or prognosis.

In embodiments, the disclosure provides a method for inhibiting proliferation of a cancer stem cell (CSC) comprising contacting the CSC with an inhibitor of CCL20 and/or an inhibitor of CCR6 in an amount effective to inhibit proliferation and/or reduce survival of the CSC. In embodiments, the method comprises administering an effective amount of a CCR6 inhibitor that is selective for CCR6. In some embodiments, the method comprises administering an effective amount of a CCL20 inhibitor that is selective for CCL20. In some embodiments the inhibitor selectively inhibits the CCR6/CCL20 biochemical cascade or axis. The term “selective” when used in connection with “inhibitor” or “inhibits” relates to a compound (e.g., a small molecule or biological molecule as described herein) that has increased inhibitory activity for a target, for example, CCR6 or CCL20, relative to the inhibitory activity for other biomolecules.

In embodiments, the disclosure relates to a method for treating a therapeutically-resistant cancer in a subject who has previously received a therapy and who is in need of treatment, comprising administering to the subject an inhibitor of CCL20 and/or an inhibitor of CCR6 in an amount effective to inhibit or kill cancer stem cells (CSCs) and/or induce differentiation in CSCs present in the therapeutically-resistant cancer.

In further embodiments, the disclosure relates to a method for treating a therapeutically-resistant cancer in a subject who has previously received a therapy and who is in need of treatment, comprising administering to the subject an inhibitor of CCL20 and/or an inhibitor of CCR6 in an amount effective to treat the therapeutically-resistant cancer.

In other embodiments, the disclosure relates to a method for treating a cancer that comprises cancer stem cells (CSCs), the method comprising administering to a subject in need of treatment an inhibitor of CCL20 and/or an inhibitor of CCR6 in an amount effective to target and inhibit or kill the CSCs and/or induce differentiation in CSCs in the cancer.

In other embodiments, the disclosure relates to a method for treating a cancer that comprises cancer stem cells (CSCs), the method comprising administering to a subject in need of treatment an inhibitor of CCL20 and/or an inhibitor of CCR6 in an amount effective to treat the cancer.

In further embodiments, the disclosure relates to a method for treating cancer in a subject who has recurring or relapsed cancer comprising administering to a subject an inhibitor of CCL20 and/or an inhibitor of CCR6 in an amount effective to inhibit or kill CSCs and/or induce differentiation in CSCs in the cancer.

In further embodiments, the disclosure relates to a method for treating cancer in a subject who has recurring or relapsed cancer comprising administering to a subject an inhibitor of CCL20 and/or an inhibitor of CCR6 in an amount effective to treat the cancer.

Other embodiments of the disclosure relate to a method for preventing a recurrence or a relapse of cancer, comprising administering to a subject in need of prevention of recurrence or relapse of cancer an inhibitor of CCL20 and/or an inhibitor of CCR6 in an amount effective to prevent recurrence or relapse of cancer.

In further embodiments, the disclosure relates to a method for reducing the risk of cancer relapse in a subject who has cancer comprising administering to the subject an inhibitor of CCL20 and/or an inhibitor of CCR6 in an amount effective to inhibit or kill CSCs and/or induce differentiation in CSCs in the cancer, thereby reducing the risk of cancer relapse in the subject.

In embodiments, the disclosure relates to a method of eliminating a cancer stem cell (CSC) comprising contacting the CSC with an inhibitor of CCL20 and/or an inhibitor of CCR6 in an amount effective to eliminate the CSC.

In some embodiments, the disclosure relates to a method of selectively reducing the number of cancer stem cells (CSCs) in a population of cancer cells comprising contacting the population of cancer cells with an inhibitor of CCL20 and/or an inhibitor of CCR6 in an amount effective to inhibit or kill CSCs and/or induce differentiation in CSCs in the population of cancer cells. Thus, the inhibitors may be administered to a subject in need of treatment and/or contacted with a number of CSCs in an amount sufficient to prohibit further proliferation of the CSCs. In some embodiments, the inhibitors may be administered in an amount effective to reduce the number of CSCs by inducing cell death (apoptosis) in the population of CSCs. In yet other embodiments, the inhibitors may be administered in an amount effective to reduce the number of CSCs by inducing cellular differentiation in the population of CSCs. Accordingly, in some embodiments the inhibitors are provided in amounts that may not necessarily induce apoptosis in the CSCs, but in amounts that are effective to reduce or deregulate the ability of CSCs to self-renew and expand the CSC cell population in a subject or a tumorigenic tissue by inducing differentiation of the CSCs. The inhibitors may also be provided in an amount that is effective to perturb a CSC “niche” such that the CSC niche is no longer able to maintain CSC and/or tumor tissue regeneration.

In some embodiments, the disclosure provides a method for reducing or inhibiting tumor growth comprising contacting the tumor with an inhibitor of CCL20 and/or an inhibitor of CCR6 in an amount effective to reduce or inhibit tumor growth. In a related aspect, the disclosure provides a method for reducing or inhibiting tumor growth in a patient comprising administering to the patient an inhibitor of CCL20 and/or an inhibitor of CCR6 in an amount effective to reduce or inhibit tumor growth.

In further embodiments, the disclosure provides a method for reducing the size of a tumor comprising contacting the tumor with an inhibitor of CCL20 and/or an inhibitor of CCR6 in an amount effective to reduce tumor size. In a related aspect, the disclosure relates to a method for reducing the size of a tumor in a patient comprising administering to the patient an inhibitor of CCL20 and/or an inhibitor of CCR6 in an amount effective to reduce tumor size.

In some embodiments, the disclosure provides a method for inhibiting, reducing, or preventing tumor invasiveness or metastasis comprising contacting the tumor with an inhibitor of CCL20 and/or an inhibitor of CCR6 in an amount effective to prevent, reduce, or inhibit tumor invasiveness or metastasis. In a related aspect, the disclosure provides a method for inhibiting, reducing, or preventing tumor invasiveness or metastasis in a patient comprising administering to the patient an inhibitor of CCL20 and/or an inhibitor of CCR6 in an amount effective to prevent, reduce, or inhibit tumor invasiveness or metastasis.

In embodiments provided by the disclosure, the methods relate to a tumor and/or a CSC, or a population of CSCs, expressing CCL20. In some embodiments, the methods relate to a tumor and/or a CSC, or a population of CSCs, expressing CCR6. In some embodiments the methods relate to a tumor and/or a CSC, or a population of CSCs, expressing CCR6 and CCL20. In further embodiments, the tumor and/or a CSC, or a population of CSCs, may comprise sphere-forming CSCs.

In various embodiments of the above aspects, the methods relate to treating a subject for a tumor disease and/or a cancer disease. In some embodiments the cancer is selected from digestive or gastro-intestinal cancers (e.g., anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric cancer; hepatocellular cancer (e.g., hepatocellular carcinoma) including adult (primary) hepatocellular cancer and childhood hepatocellular cancer; pancreatic cancer including childhood pancreatic cancer; sarcoma, rhabdomyosarcoma; islet cell pancreatic cancer; rectal cancer; and small intestine cancer); lung cancer (e.g., non-small cell lung cancer (NSCLC) and small cell lung cancer (SCLC)); head and neck cancer (e.g., lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer); ovarian and breast cancer.

As used herein, the term “subject” is intended to include human and non-human animals, particularly mammals. Examples of subjects include human subjects for example a human patient having a disorder, e.g., a disorder described herein, such as cancer, or a normal subject. A “non-human animal” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.). In particular embodiments, the subject is a human patient.

“Treatment” or “treat” refers to both therapeutic treatment and prophylactic or preventative measures. Those subjects in need of treatment include those already with the disorder as well as those prone to have the disorder or those in which the disorder is to be prevented. When used with reference to a disease or a subject in need of treatment the terms accordingly include, but are not limited to, halting or slowing of disease progression, remission of disease, prophylaxis of symptoms, reduction in disease and/or symptom severity, or reduction in disease length as compared to an untreated subject. In embodiments, the methods of treatment can abate one or more clinical indications of the particular disease being treated. Certain embodiments relating to methods of treating a disease or condition associated with CSCs that express CCL20 and/or CCR6, as well as CSCs having an activated CCR6/CCL20 cascade or axis comprise administration of therapeutically effective amounts of a compound that inhibits CCR6, CCL20, or both CCR6 and CCL20, as well as pharmaceutical compositions thereof. In embodiments, the method of treating can relate to any method that prevents further progression of the disease and/or symptoms, eliminates the disease, slows or reduces the further progression of the disease and/or symptoms, or reverses the disease and/or clinical symptoms associated with CSCs and cancers associated with CSCs that express CCR6 and/or CCL20.

In some embodiments the methods may comprise a therapeutically effective amount of an agent that is sufficient to stop or slow the progression of cancer. In further embodiments, a therapeutically effective amount is an amount sufficient to reduce the number of cancer cells, including CSCs, in the subject (e.g., killing of cancer cells and/or inhibiting proliferation of cancer cells and/or inducing differentiation of CSCs). Methods for monitoring the proliferation of cancer cells and progress of cancer in a subject (e.g., tumor size, cell counts, biochemical markers, secondary indications, etc.) are discussed herein and may also include techniques generally known in the art.

As discussed herein, some embodiments provide a method of treatment that comprises administering an inhibitor in conjunction with radiation, surgery, immunotherapy, or other chemotherapeutics. In some embodiments, the method includes administration of a therapeutically effective amount of an inhibitor of CCL20 in combination with an additional anti-cancer agent. In other embodiments, the method includes administration of a therapeutically effective amount of an inhibitor of CCR6 in combination with an additional anti-cancer agent. A wide variety of anti-cancer (i.e., anti-neoplastic) agents are known in the art and include, for example alkylating agents, antimetabolites, natural antineoplastic agents, hormonal antineoplastic agents, angiogenesis inhibitors, differentiating reagents, RNA inhibitors, antibodies or immunotherapeutic agents, gene therapy agents, small molecule enzymatic inhibitors, biological response modifiers, microtubule inhibitors, DNA damage response inhibitors, anti-metastatic agents and other chemotherapeutic agents.

“Administration” or “administering,” as used herein, refers to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

“Co-administered,” as used herein, refers to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks or one month of each other. Suitably, compounds or agents are co-administered during the period in which each of the compounds or agents are exerting at least some physiological effect and/or has remaining efficacy.

“Contacting,” as used herein as in “contacting a cell,” refers to contacting a cell directly or indirectly in vitro, ex vivo, or in vivo (i.e. within a subject, such as a mammal, including humans, mice, rats, rabbits, cats, and dogs). Contacting a cell, which may also include “reacting” a cell with or “exposing” a cell to an inhibitor compound, can occur as a result of general administration of a compound or agent to a subject or addition or application of the inhibitor to a vessel containing a cell or tissue or a fluid containing a cell or tissue. Thus, contacting the cell with an inhibitor may refer to administration of an inhibitor to a subject, tissue, or region of a subject or tissue that comprises a cell (e.g., a CSC cell in a localized region or a system in a subject (e.g., lymphatic, circulatory, etc.)) and may not physically contact a target cell. Contacting encompasses the administration to a cell, tissue, mammal, subject, patient, or human. Further, contacting a cell includes adding an agent to a cell culture. Other suitable methods may include introducing or administering an agent to a cell, tissue, mammal, subject, or patient using appropriate procedures and routes of administration as discussed herein or otherwise known in the art.

The disclosure also relates to one or more uses of an inhibitor of CCL20 and/or an inhibitor of CCR6, or a combination thereof in a composition, including therapeutic and pharmaceutical compositions. In embodiments, the uses and compositions comprise the inhibitor(s) in amounts effective to provide for the various methods disclosed herein. In embodiments, the one or more uses relate to uses for the manufacture of a medicament for treating (e.g., killing, eliminating, inhibiting, reducing the progression and/or rate of progression of cell proliferation, reducing the self-renewal and/or expansion capacity of cells (CSCs), reducing the progression and/or rate of progression of a disease state) one or more of the tumor types, cancer stem cells, and/or cancers described herein.

### Detection Methods

In some aspects and embodiments the methods disclosed herein provide for and/or comprise one or more methods useful in the detection, identification, and/or quantification of a cancer stem cell (CSC). In some embodiments the method comprises detecting, identifying, and/or quantifying the amount of CCL20 and/or CCR6 in a biological sample comprising CSCs. In some embodiments, the methods provide for diagnosis, prognosis, quantification, identification, and/or detection of the presence of a cancer stem cell (CSC) in a sample comprising cancer cells, the method can comprise one or more of:
contacting the sample with an agent that binds to a CCL20 nucleic acid sequence or a CCL20 amino acid sequence;
detecting the presence or absence of binding between the agent and the CCL20 nucleic acid sequence or the CCL20 amino acid sequence; and/or
identifying the presence of the CSC in the sample upon detection of binding between the agent and the CCL20 nucleic acid sequence or the CCL20 amino acid sequence.

In some embodiments, the methods provide for diagnosis, prognosis, quantification, identification, and/or detection of the presence of a cancer stem cell (CSC) in a sample comprising cancer cells, the method can comprise one or more of:
contacting the sample with an agent that binds to a CCR6 nucleic acid sequence or a CCR6 amino acid sequence;
detecting the presence or absence of binding between the agent and the CCR6 nucleic acid sequence or the CCR6 amino acid sequence; and/or
identifying the presence of the CSC in the sample upon detection of binding between the agent and the CCR6 nucleic acid sequence or the CCR6 amino acid sequence.

In these methods the steps may be used alone or in any series of combinations with each other or other techniques generally known in the art. In some embodiments of this aspect, the method can further comprise one or more of:
quantifying the amount of the CCL20 and/or CCR6 nucleic acid sequence or the CCL20 and/or CCR6 amino acid sequence in the sample;
comparing the amount of the CCL20 and/or CCR6 nucleic acid sequence or the CCL20 and/or CCR6 amino acid sequence to a reference level of CCL20 and/or CCR6 nucleic acid or CCL20 and/or CCR6 amino acid; and/or
identifying the presence of the CSC in the sample when the detected amount is greater than the reference level.

In yet further embodiments, the method may comprise an agent comprising a detectable moiety. In some embodiments, the agent may comprise a nucleic acid sequence that hybridizes to at least a portion of the CCL20 and/or CCR6 nucleic acid sequence under stringent hybridization conditions. In some embodiments, the agent may comprise an antibody that specifically binds to at least a portion of the CCL20 and/or CCR6 amino acid sequence.

In various embodiments of the above aspects, the methods relate to a tumor and/or a CSC, or a population of CSCs, expressing CCL20. In some embodiments of the above aspects, the methods relate to a tumor and/or a CSC, or a population of CSCs, expressing CCR6. In further embodiments, the tumor and/or a CSC, or a population of CSCs, may comprise sphere-forming CSCs.

In addition to the methods described herein that are useful for detecting, identifying, and/or quantifying a CSC in a sample, these aspects and embodiments can also include any method and technique that is available to one of ordinary skill in the art. As non-limiting examples, CSCs may be identified, detected, and isolated using techniques including flow cytometry based on cell surface markers that are expressed in, or specific for particular CSCs; detection of side-population (SP) phenotypes by dye exclusion (e.g., Hoechst 33342, as disclosed in Moserle, L., et al., Cancer Res. (2008) 68; 5658-5668); ability to grow/proliferate as floating spheres in serum-free medium (e.g., disclosed herein as well as in Ryback, A. P., et al., Biochim. Biophys. Acta (2011) 1813; 683-694); and determination of particular enzymatic activity such as, for example, aldehyde dehydrogenase activity, and levels of polycomb markers including polycomb heterochromatin marker, H3K27me³ levels, and polycomb group protein enhancer of zeste 2 (EZH2).

In some embodiments, for example, elevated aldehyde dehydrogenase (ALDH) expression and activity has been reported in cancer precursor cells of various lineages, (e.g., hematopoietic, mammary, endothelial, mesenchymal, neural) and techniques and commercially available kits (e.g., ALDEFLUOR™, Stemcell Technologies) may be used in connection with the various aspects and embodiments herein. Similarly, while there are currently no universally expressed cell surface markers for identifying CSCs, a number of surface markers have been associated with CSCs and associated tumor types, including, for example, ALDH, CD13, CD15, CD24, CD44, CD90, CD117, CD133, CD166, CD326, (see, e.g., Xia, P., Curr Stem Cell Res Ther. (2014) March; 9(2): 102-11; Shimamura, M., et al., Endocr. J., (2014) 61(5):481-90; Tirino, V., et al., FASEB J., (2013) January; 27(1):13-24).

In some embodiments, because of the lack of one or more universal surface markers for the detection or identification of a CSC in a sample, methods of detecting, identifying, and/or quantifying comprise cell culture techniques comprising sphere cultures and growth assays such as disclosed herein or otherwise known in the art.

### B. Inhibitors

The methods, uses, and compositions described herein include embodiments relating to agents capable of inhibiting, downregulating, or abolishing the activity and/or the expression of CCR6 and/or CCL20, or any combination of one or more such inhibitor agents. As long as the agent possesses the inhibitory function (e.g., inhibits CCR6, CCL20, or both CCR6 and CCL20 expression and/or activity), the inhibitor agent may be selected from any class of compound. For example, the inhibitors may be selected from the group consisting of a polypeptide that inhibits CCR6 and/or CCL20, a small molecule that inhibits CCR6 and/or CCL20, a polynucleotide that inhibits CCR6 and/or CCL20, an aptamer that inhibits CCR6 and/or CCL20, an antibody that inhibits CCR6 and/or CCL20, or an antisense molecule or siRNA molecule that inhibits CCR6 and/or CCL20. Thus, the inhibitors as used herein refer to any compound that reduces, inhibits, downregulates, or abolishes the expression and/or function of the relevant molecule (e.g., CCR6 and/or CCL20), or an agent suitable for neutralizing, reducing, or inhibiting the expression or function of the molecule. The inhibitors described herein may exert action by any mechanism including, for example, binding to CCR6 and/or CCL20. Upon binding, an inhibitor may, for example, inhibit the interaction of CCR6 and/or CCL20 with a receptor or a ligand, such that CCR6 or CCL20 cannot activate the receptor, or cannot be activated by a ligand. In other embodiments, the agent(s) capable of inhibiting the activity of CCR6 and/or CCL20 may inhibit the chemotactic activity, may sequester the proteins and reduce bioavailability, or any combinations thereof.

In some embodiments, the inhibitor may be a polypeptide or a small molecule capable of binding to CCR6 or CCL20, or both, and may be identified by any technique routine in the art (e.g., screening small compound or polypeptide libraries). A “small molecule” as used herein generally refers to small organic compounds having low molecular weight. A small molecule may be a synthetic compound not known to occur in nature or a naturally-occurring compound isolated from or known to occur in natural sources, such as e.g. cells, plants, fungi, animals and the like. In some embodiments, a small molecule may have a molecular weight of less than 5000 Daltons, less than 4000 Daltons, less than 3000 Daltons, less than 2000 Daltons, less than 1000 Daltons, or less than about 800 Daltons. In such embodiments a small molecule typically has a molecular weight of greater than about 100 Daltons.

Whether a small molecule or polypeptide is capable of binding to CCR6 or CCL20 may be determined by any routine technique or assay, as well as the techniques and assays that are described herein. For example, techniques may include a yeast two-hybrid assay or a biochemical assay such as e.g. a pull-down assay, a co-immunoprecipitation assay, an enzyme-linked immunosorbent assay (ELISA), a quantitative radioligand binding assay, a Plasmon resonance assay or any other method routinely used in the art. Typically, when using pull-down or Plasmon resonance assays, it is useful to fuse or otherwise link at least one of the proteins to an affinity tag such as HIS-tag, GST-tag or other detectable moiety generally used in the art. Whether a polypeptide or any other compound to be tested is capable of inhibiting the activity of CCR6 and/or CCL20 may be determined by measuring the activity of membrane-bound polypeptide. Suitably, a polypeptide is capable of inhibiting the activity of CCR6 and/or CCL20.

In embodiments, the inhibitor may be an “aptamer” which refers to a DNA, RNA or peptide aptamer having specificity for CCR6 or for CCL20. A polynucleotide aptamer comprises anywhere from about 10 to about 300 nucleotides in length. Typically, an aptamer ranges from about 30 to about 100 nucleotides in length, and in some embodiments may range from about 10 to 60 nucleotides in length. Aptamers may be prepared by any known method, including synthetic, recombinant, and purification methods, and may be used alone or in combination with other aptamers specific for CCR6 or for CCL20.

In some embodiments, the inhibitor may be an antibody that specifically binds CCR6 or CCL20. In some embodiments the antibody may be a monoclonal or polyclonal antibody, or a polyspecific antisera. In such embodiments, the antibody may also include antibody variants or fragments such as, for example, single chain antibodies, diabodies, minibodies, single chain Fv fragments (sc(Fv)), sc(Fv)₂ antibodies, Fab fragments, or F(ab′)₂ fragments, as long as the variant or fragment retains specific binding properties to the target (e.g., CCR6 or CCL20).

In other embodiments, the inhibitor may be an antisense molecule comprising a polynucleotide which is complementary to at least a portion of CCR6 mRNA, or CCL20 mRNA, or both. In some embodiments, the antisense molecule is suitable for use in methods that inhibits translation of mRNA in a cell. The antisense molecule may comprise DNA, RNA, or both DNA and RNA, as well as chemically modified nucleic acids. Further, in some embodiments the antisense molecule may be single stranded or double stranded. An antisense molecule may comprise about 10 to about 500 nucleotides and, in some embodiments may have any number of lengths, for example, ranging from about 11 to about 200 nucleotides, about 12 to about 100 nucleotides, about 13 to about 75 nucleotides, about 14 to about 50 nucleotides, about 15 to about 40 nucleotides, about 16 to about 30 nucleotides, or about 17 to about 25 nucleotides. One of ordinary skill in the art will appreciate that these ranges represent illustrative embodiments.

In some embodiments, the inhibitor may comprise a siRNA molecule that reduces or inhibits the expression of CCR6 or CCL20, or both CCR6 and CCL20. The siRNA molecule can, in some embodiments, be a single stranded or double stranded siRNA molecule that comprises a sequence capable of hybridizing to CCR6 mRNA, CCL20 mRNA, or both, and induce RNA interference or another antisense mechanism that reduces or inhibits expression of protein. siRNA molecules may be of any sequence that allows the siRNA molecule to induce RNA interference resulting in reduction or inhibition of the expression of CCR6 protein or CCL20 protein. The siRNA molecule may, in some embodiments have a length of between 10 and 100, between 12 and 80, between 14 and 60, between 16 and 50, between 17 and 40. Suitably the siRNA has a length ranging from 18 to 30 nucleotides and, in certain embodiments between about 18 and about 26 nucleotides.

In embodiments comprising an inhibitory nucleic acid molecule, such inhibitors can bind to a target CCR6 or CCL20 nucleic acid sequence under stringent binding conditions. The terms “stringent conditions,” “stringent binding conditions,” or “stringent hybridization conditions” refers to conditions under which a polynucleotide will hybridize to a target sequence, to a detectably greater degree than other sequences (e.g., at least 2-fold over background). One non-limiting example of stringent conditions include those in which hybridization in 50% formamide, 1 M NaCl, 1% SDS at 37° C., and a wash in 0.1×SSC at 60° C. to 65° C. is performed.

Given the polynucleotide sequence of CCR6 or CCL20, an inhibitory nucleic acid molecule can be designed using motifs and targeted to a region that may be predicted to be effective for inhibitory activity, using standard techniques in the art.

Alternatively or additionally any further methods which are suited to determine the activity of CCR6 and/or CCL20 and which are available to one of skill in the art may be used. The inhibitors useful in the various aspects and embodiments described herein may inhibit the activity of CCR6 and/or CCL20 by at least 10%, at least 20%, at least 30%, at least 40%, at least 50%, at least 60%, at least 70%, more preferably at least 80%, at least 90%, at least 91%, at least 92%, at least 93%, at least 94%, at least 95%, at least 96%, at least 97%, at least 98% or at least 99% when compared to a control. In particular embodiments, the inhibitors are effective to inhibit the activity of CCR6 and/or CCL20 by at least 50%, at least 60% or at least 70%. In other embodiments the inhibitors may inhibit the activity of CCR6 and/or CCL20, by at least 85% to about 100%, at least 90% to about 100%, at least 95% to about 100%, or by about 100%.

In embodiments, the inhibitors may be combined in a composition that comprises one or more further active agents suitable for the treatment or prevention of a tumor disease and/or a cancer. Some non-limiting examples of such active compounds including chemotherapeutic agents generally known in the art such as, for example, temozolomide, adriamycin, doxorubicin, epirubicin, 5-fluorouracil, cytosine arabinoside (“Ara-C”), cyclophosphamide, thiotepa, busulfan, cytoxin, taxoids, (e.g., paclitaxel), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, caminomycin, aminopterin, dactinomycin, mitomycins, melphalan and other related nitrogen mustards and hormonal agents that act to regulate or inhibit hormone action on tumors such as tamoxifen and onapristone.

### C. Pharmaceutical Compositions and Formulations

In some aspects, the disclosure provides pharmaceutical compositions. Such pharmaceutical compositions may be compositions comprising an inhibitor of CCL20, an inhibitor of CCR6, a combination of an inhibitor of CCL20 and CCR6, or any other combinations thereof. Such pharmaceutical compositions may also be compositions comprising a pharmaceutically acceptable excipient. In certain aspects, the pharmaceutical compositions of the disclosure are used as a medicament.

In certain aspects, the inhibitors disclosed herein may be formulated with a pharmaceutically acceptable carrier, excipient or stabilizer, as pharmaceutical compositions. In certain aspects, such pharmaceutical compositions are suitable for administration to a human or non-human animal via any one or more routes of administration using methods known in the art. As will be appreciated by the skilled artisan, the route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium and the like. These and additional known pharmaceutical carriers, excipients and/or additives suitable for use in the formulations described herein are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be selected that are suitable for the mode of administration, solubility and/or stability desired or required.

In one aspect, the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). In certain specific aspects, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

When used for in vivo administration, the formulations of the disclosure should be sterile. The formulations of the disclosure may be sterilized by various sterilization methods, including sterile filtration, radiation, etc. In one aspect, the formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005).

In embodiments of this aspect, therapeutic compositions can be formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Formulations of the present disclosure which are suitable for topical or transdermal administration include powders, sprays, ointments, pastes, creams, lotions, gels, solutions, patches and inhalants. The inhibitors and other actives may be mixed under sterile conditions with a pharmaceutically acceptable carrier, and with any preservatives, buffers, or propellants which may be required (see, e.g., U.S. Pat. Nos. 7,378,110; 7,258,873; 7,135,180; US Publication No. 2004-0042972; and 2004-0042971).

The formulations may conveniently be presented in unit dosage form and may be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration. As noted above, any commonly used dosing regimen (e.g., 1-10 mg/kg administered by injection or infusion daily or twice a week) may be adapted and suitable in the methods relating to treating human cancer patients.

The disclosure similarly contemplates that formulations suitable for diagnostic and research use may also be made. The concentration of active agent in such formulations as well as the presence or absence of excipients and/or pyrogens can be selected based on the particular application and intended use.

### D. Kits

Another aspect of the disclosure relates to a kit. In one aspect, a kit comprises any of the inhibitors, reagents, compositions, or pharmaceutical compositions as described above, and instructions or a label directing appropriate use or administration. Optionally, a kit may also include one or more containers and/or a syringe or other device to facilitate delivery or use. The disclosure contemplates that all or any subset of the components for conducting research assays, diagnostic assays and/or for administering therapeutically effective amounts may be enclosed in the kit. Similarly, the kit may include instructions for making a conjugate by, for example forming a covalent bond between an inhibitor of the disclosure and a therapeutic or diagnostic moiety under suitable conditions to form a conjugate. By way of additional example, a kit for use in a therapeutic method of the disclosure may comprise a solution containing a pharmaceutical formulation of the inhibitor(s) of CCL20 or/and CCR6, or a lyophilized preparation of one or more inhibitors, and instructions for administering the composition to a patient in need thereof and/or for reconstituting the lyophilized product.

The present disclosure also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In certain aspects, the formulation is suitable for intravenous administration, such as for intravenous infusion to a human or animal.

In a specific aspect, the formulations of the disclosure are formulated in single dose vials as a sterile liquid. Exemplary containers include, but are not limited to, vials, bottles, pre-filled syringes, IV bags, blister packs (comprising one or more pills). Optionally associated with such container(s) can be a notice in the form prescribed by a governmental agency regulating the manufacture, use or sale of pharmaceuticals or biological products, which notice reflects approval by the agency of manufacture, use or sale for human diagnosis and/or administration.

As with any pharmaceutical product, the packaging material and container are designed to protect the stability of the product during storage and shipment. Further, the products of the disclosure include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, etc., and other monitoring information.

A kit for diagnostic assays may comprise a solution containing an inhibitor of CCL20 and/or CCR6 as disclosed herein, or alternatively or in addition, reagents for detecting the presence of CCL20 and/or CCR6. The various reagents may be labeled according to methods known in the art and described herein, including but not limited to labels such as small molecule fluorescent tags, proteins such as biotin, GFP or other fluorescent proteins, or epitope sequences such as his or myc. Similarly, primary antibodies used for detecting CCL20 and/or CCR6 may be included in the kit. Primary antibodies may be directed to sequences on CCL20 and/or CCR6 or to labels, tags, or epitopes with which the CCL20 and/or CCR6 may be labeled. Primary antibodies may, in turn, be labeled for detection, or, if further amplification of the signal is desired, the primary antibodies may be detected by secondary antibodies, which may also be included in the kit.

Kits for research use are also contemplated. Such kits may, for example, resemble kits intended for diagnostic or therapeutic uses but further include a label specifying that the kit and its use is restricted to research purposes only.

### Labels, Conjugates and Moieties

The disclosure relates to methods, compositions, and kits that comprise reagents that may be conjugated to labels for the purposes of diagnostics and other assays wherein one or more target molecules may be bound and detected by such labeled reagent(s). Labels include, without limitation, a chromophore, a fluorophore, a fluorescent protein, a phosphorescent dye, a tandem dye, a particle, a hapten, an enzyme and a radioisotope.

In certain aspects, the reagents are conjugated to a fluorophore. The choice of the fluorophore attached to the reagent(s) will determine the absorption and fluorescence emission properties of the conjugated molecule. Physical properties of a fluorophore label that can be used include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. Other suitable properties of the fluorescent label may include cell permeability and low toxicity, for example if labeling of the target(s) is to be performed in a cell or an organism (e.g., a living animal).

In certain aspects, an enzyme is a label and is conjugated to a reagent. Enzymes may be suitable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art and include for example, oxidoreductases such as horseradish peroxidase and a substrate such as 3,3′-diaminobenzidine (DAB); phosphatase enzymes such as an acid phosphatase, alkaline and a substrate such as 5-bromo-6-chloro-3-indolyl phosphate (BCIP); glycosidases, such as beta-galactosidase, beta-glucuronidase or beta-glucosidase and a substrate such as 5-bromo-4-chloro-3-indolyl beta-D-galactopyranoside (X-gal); additional enzymes include hydrolases such as cholinesterases and peptidases, oxidases such as glucose oxidase and cytochrome oxidases, and reductases for which suitable substrates are known.

Enzymes and their appropriate substrates that produce chemiluminescence are suitable for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another aspect, haptens such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain aspects, fluorescent proteins may be conjugated to the reagent(s) as a label. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra.

In certain aspects, the label is a radioactive isotope. Examples of suitable radioactive materials include, but are not limited to, iodine (121I, 123I, 125I, 131I), carbon (14C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁶⁸Ga, ⁶⁷Ga), palladium (¹⁹³Pd), molybdenum (⁹⁹Mo), xenon (¹³⁵Xe), fluorine (¹⁸F), ¹⁵³SM, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh and ⁹⁷Ru.

In some aspects, drugs or toxins may be conjugated to the reagent(s). For example, a reagent may be conjugated to a cytotoxic drug. In this example, the reagent may bind to a target antigen on a cell surface such as, for example CCR6, or a complex between CCR6 and CCL20, and the cytotoxic drug is then delivered to the cell. In some embodiments, the reagent-conjugate may be internalized in the cell, which releases the cytotoxic drug to the cell. Any cytotoxic drug known in the art may be conjugated. Some antibody conjugates are already approved by the FDA or are currently undergoing clinical trials.

The Examples that follow are presented in order to provide additional illustration of some of the aspects and embodiments disclosed above. The Examples should not be construed in any way as limiting to the breadth of the scope of the disclosure or the appended claims.

### EXAMPLES

Materials & Methods

Monolayer and sphere culture. For cell culture experiments, a panel of eight breast cancer cell lines spanning various breast cancer subtypes, MDAMB468, HCC1937, SUM159PT, MCF7, BT549, BT474, T47D, and Hs578T, a panel of four pancreatic cancer cell lines, Aspc1, Bxpc3, HPAC, and Panc1, and a panel of seven colorectal cancer cell lines, LS411N, SW1417, NCI-H508, Colo205, HT29, HCT116, Lovo, were maintained in humidified incubators at 37° C., 5% CO₂, in suppliers recommended media. All cell lines were originally obtained from ATCC (Manassas, Va.), with the exception of SUM159PT, which was obtained from Asterand (Detroit, Mich.).

For standard monolayer tissue culture, cells were harvested with 0.25% Trypsin-EDTA (Life Technologies, Grand Island, N.Y.), washed three times in Hank's Balanced Salt Solution (HBSS, Life Technologies), and plated in full serum media to tissue culture treated plates. For sphere culture, cells were harvested with 0.25% Trypsin-EDTA, washed three times in Hank's Balanced Salt Solution, and plated in serum-free defined medium, supplemented with epidermal growth factor (EGF), basic fibroblast growth factor (bFGF), insulin, bovine serum albumin (BSA), and Knockout serum (Life Technologies), to ultra-low attachment tissue culture plates (Corning).

Cell growth in monolayer assays were read after a 4 day incubation period by CellTiter Glo® (CTG) luminescent cell viability assay (Promega, Madison, Wis.) according to suppliers recommendation. Sphere growth assays were read after a 4 day incubation period by direct counting of sphere colonies using High Content Imaging (Arrayscan VTI, Thermo Scientific, Waltham, Mass.), as well as by CTG assay.

CCR6-based cell isolation. CCR6-positive (CCR6⁺) were separated from CCR6-negative (CCR6⁻) cells in cell lines by magnetic bead separation. A tetrameric antibody complex (TAC) positive selection system was developed by complexing a mouse-anti-human CCR6 monoclonal antibody (BD Biosciences, San Jose, Calif.) with the “Do-It-Yourself” EasySep Selection kit (Stem Cell Technologies, Vancouver, BC, Canada). Use of this positive selection kit according to suppliers recommended protocol captured CCR6⁺ cells by magnetic bead isolation, while CCR6⁻ cells were retained in the unbound supernatant. Isolated CCR6⁺ and CCR6⁻ cells were then plated to monolayer culture and stimulated with recombinant human CCL20 (rhCCL20) as described in other sections.

Enzyme-Linked ImmunoSorbent Assay (ELISA).

CCL20 levels secreted into conditioned medium of cultured cells were measured at day 4 of culture using a commercially obtained sandwich ELISA (R&D systems, Minneapolis, Minn.) according to supplier's protocol. CCL20 levels were directly measured in picogram/milliliter (pg/mL), interpolated from a standard curve. Secreted CCL20 levels were normalized to and reported as X fold-change compared to monolayer cultured cells from the same line.

CCL20 Stimulation and Inhibition.

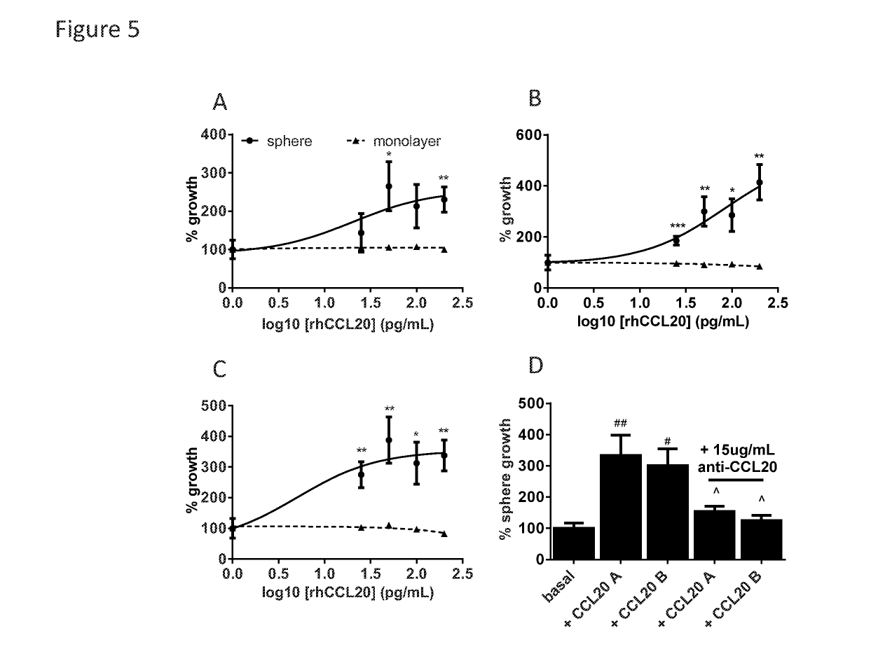

Commercially obtained recombinant human CCL20 (rhCCL20 or CCL20) sourced from R&D systems was used for all studies, with exception of data depicted in FIG. 5D where recombinant human CCL20 was also obtained from Life Technologies for comparison. Isotype control and anti-CCL20 monoclonal antibodies were also commercially sourced (R&D systems). All materials were reconstituted and stored according to manufacturer's recommendation.

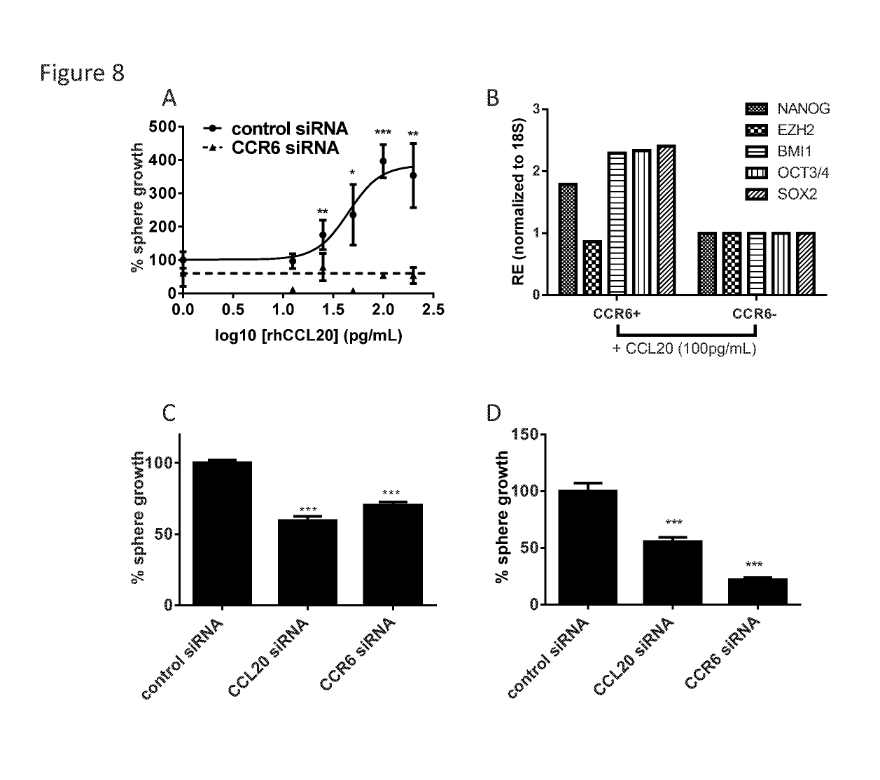

For stimulation and inhibition studies, cells were plated for monolayer or sphere assay as described above. At the time of plating, cells were treated with rhCCL20 or antibody treatments at doses described in the experiments and results. For FIG. 8A, cells were transfected with 20 nM CCR6 siRNA or non-target control siRNA (pooled constructs, obtained from Santa Cruz Biotechnology, Santa Cruz, Calif.) for 48 hours prior to addition of rhCCL20, while for FIG. 8C, 8D, cells were transfected with 20 nM CCL20, CCR6, or non-target control siRNA (pooled constructs, Santa Cruz Biotechnology) at the time of plating, concomitant with antibody treatment of the cells. Transfection of siRNA was performed using lipid (Lipofectamine RNAimax (Life Technologies)) as the carrier, and complexed to siRNA at a ratio of 1:2.5 siRNA:lipid. All treatments ran for 4 days undisturbed, after which cells were transferred and prepared for monolayer/sphere assays, flow cytometry analysis, or RNA extraction as described in previous or later sections.

Care and use of patient-derived Xenograft animal models. All animals were housed under 12:12 hour light:dark cycle, with ad libitum access to water and chow, and allowed to acclimatize for at least one week prior to study initiation. All animal studies were approved by MedImmune's Institutional Animal Care and Use Committee.

The BR_PDX_1 and PA_PDX_1 models were obtained from Jackson Laboratories as female NSG mice bearing subcutaneous primary human pancreatic tumor xenografts at passage P1 (PA_PDX_1) or passage P2 (BR_PDX_1). The PA_PDX_2 model was obtained from Asterand as primary human pancreatic adenocarcinoma resections. All samples were obtained under informed patient consent, in accordance with their respective IRBs, which we reviewed and approved according to MedImmune's Human Biological Sample Acquisition policy.

All tumor material was propagated by subcutaneous implantation of tumor chunks into the hind flank of six to eight week old female NSG mice (NOD.Cg-Prkdc^{scid} Il2rg^{tmlWjl}/SzJ, Jackson Laboratories) or female Beige Nude XID mice (Lyst^{bg}Foxn1^{nu}Btk^{xid}, Envigo) under sterile conditions. Tumors were passaged by this methodology for up to passage 6 (P6), whereby passage 0 (P0) is considered the first xenograft established from the patient biopsy.

When tumors reached approximately 750 mm³, they were harvested, washed in Hank's Balanced Salt Solution (HBSS), minced with a sterile razor-blade, and dissociated to single cell suspension in 20 U/mL collagenase III (Worthington Biochemical). Dissociated tumor cells were used in fluorescence activated cell sorting as described below.

For the in vivo tumorigenicity study, Panc1 cells were cultured to sphere forming conditions, under treatment with 10 μg/mL CCL20 IgG or isotype IgG as control started at the time of plating. Following a 4 day culture, spheres were dissociated in StemPro Accutase to single cell suspension. Dissociated sphere cells were mixed 1:1 with Matrigel GFR (Corning) and injected subcutaneously into the hind flank of 6-8 week old female nu/nu mice (Hsd:Athymic Nude-Foxn1^{nu}, Envigo) at 30,000 cells per mouse. Tumor volume was monitored and measured over time, whereby time-to-tumor-take was recorded as the first observation of a palpable tumor.

Flow Cytometry and Fluorescence Activated Cell Sorting (FACS).

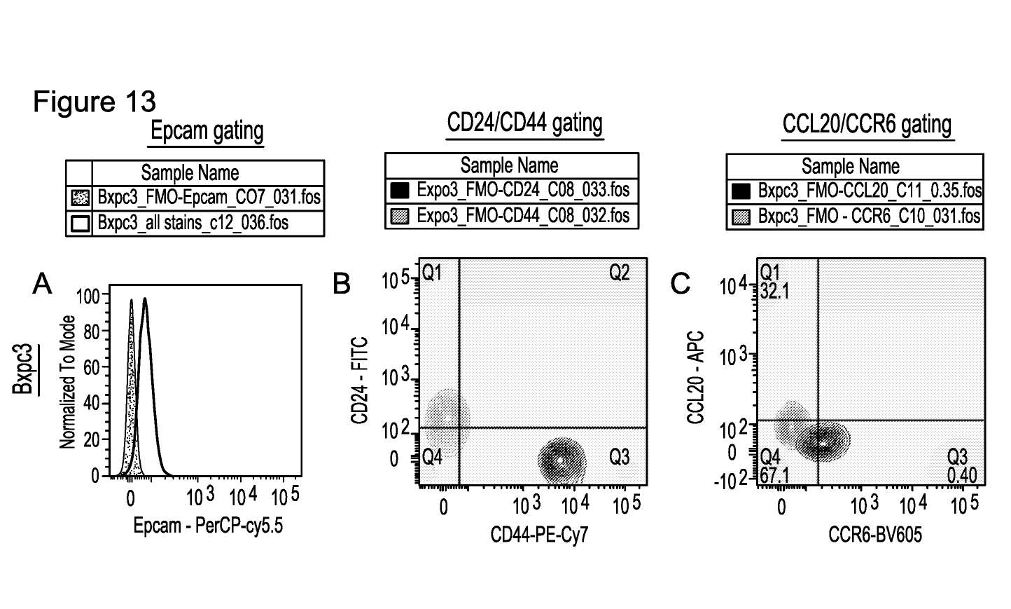

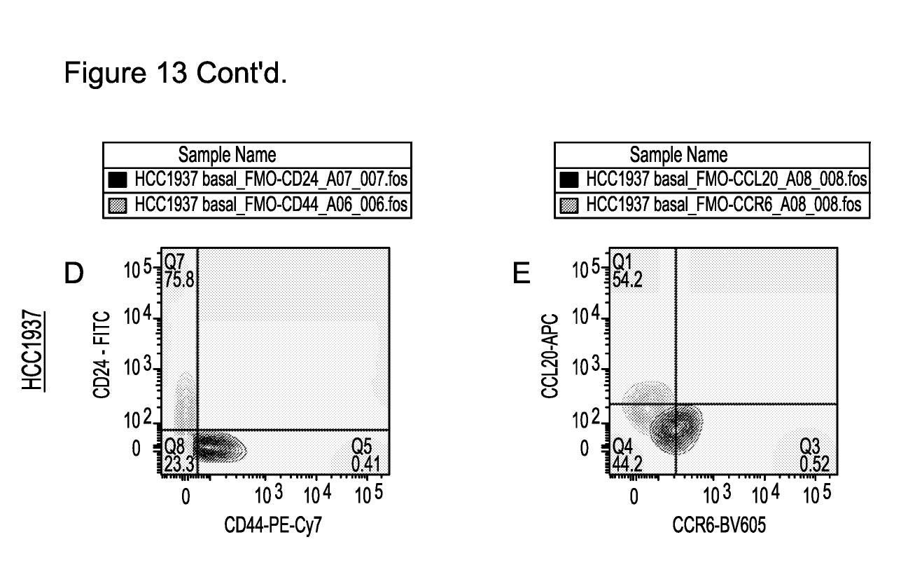

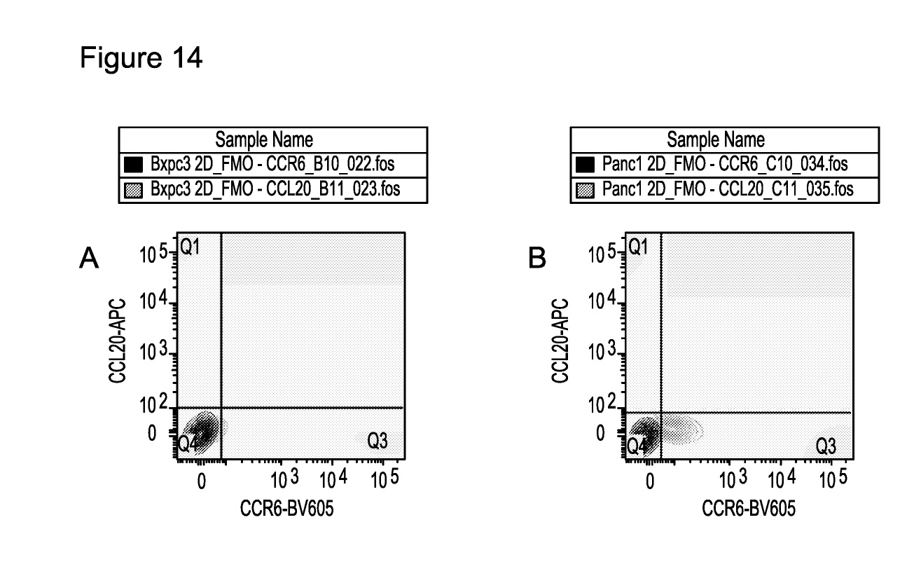

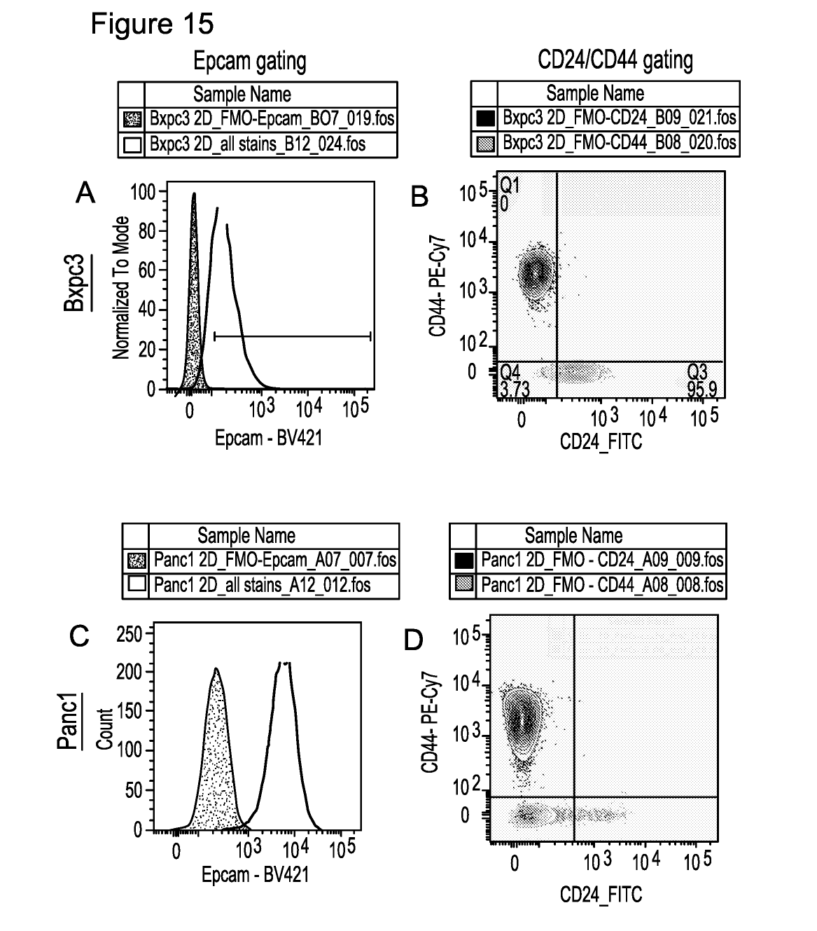

Samples were prepared for flow cytometry experiments by incubating live cells with Golgistop (BD Biosciences) for 1 hour at 37° C. to arrest CCL20 secretion. Following this, cells were harvested with an accutase cell dissociation solution (Life Technologies), washed and resuspended at approximately 2.5×10⁶ cells per mL into Hank's Balanced Salt Solution (HBSS). Cells were stained with a multicolor panel consisting of all or several of the following antibodies: anti-human CCL20-APC (R&D systems), anti-human CCR6-BV605, anti-human CD24-FITC, anti-human CD44-PE-Cy7, and anti-human Epcam-PerCP-Cy5.5 or Epcam-BV421 (all from BD Biosciences). Primary tumor xenografts were additionally stained with anti-mouse H2Kd-PE antibody (BD Biosciences) to allow all tumor-associated mouse endothelial and stromal cells to be excluded from subsequent analyses. In experiments containing CCL20 staining, cells were stained for all surface receptors prior to fixing and permeabilizing them for anti-CCL20 staining. Single staining controls were used to define compensation matrices, and full-minus-one staining controls were used to define gates. For cytometry experiments on live cells, DAPI (Invitrogen) was added to each sample at 30 μM to gate out dead cells. Standard flow cytometry experiments were run on an LSRII with 4-laser configuration, while cell sorting experiments were run on a FACSAriaII (BD Biosciences). FACS gating was determined by fluorescence-minus-one (FMO) staining as shown in FIG. 13, FIG. 14, and FIG. 15.

Quantitative RT-PCR.

All samples were processed for RNA extraction using the Ambion RNAaqueous kit (Applied Biosystems). RNA yield and quality was determined by UV spectrophotometry at 260/280 nm. From 10-100 nanograms of mRNA of each sample was subjected to reverse transcription PCR (RT-PCR) at 42° C. in the presence of random primer oligodeoxyribonucleotides (Life Technologies) and Superscript III reverse transcriptase (Life Technologies). Following RT-PCR, the 20 ng cDNA product was pre-amplified for 14 cycles using pooled Taqman gene expression assays (Life Technologies), each at 0.2× concentration, for the following genes:

TABLE 1

| Gene symbol | Taqman assay ID |
|---|---|
| 18S rRNA | 4310893E |
| SOX2 | Hs01053049_s1 |
| NANOG | Hs02387400_g1 |
| OCT3/4 (POUF1) | Hs04195369_s1 |
| BMI1 | Hs00180411_m1 |
| EZH2 | Hs01016789_m1 |
| CCL20 | Hs01011368_m1 |
| CCR6 | Hs01890706_s1 |

Preamplified material for each sample was diluted 20-fold in deionized water (DI-H₂O) and subjected to quantitative PCR (qPCR) analysis using the above listed primes in technical replicates on a 7900HT Thermocycler (Life Technologies) according to supplier's recommended protocol.

Ct values were obtained from the qPCR run using SDS 2.4 software (Life Technologies), and converted to Relative Expression (RE) values by the comparative Ct method, using the formula RE=2^{-ΔΔCt}, where

ΔCt=normalized Ct=gene Ct−18S Ct

ΔΔCt=normalized sample Ct−normalized control Ct

An average±standard error of the mean was determined for RE values amongst technical replicates, and subsequently plotted as the final data representation.

TR1 Culture and Flow Cytometry Analysis.

Human naïve CD4+ T cells were freshly isolated from healthy human donor blood using the naïve CD4 T cell isolation kit II according to suppliers protocol (Miltenyi Biotech) and skewed in culture towards TR1 cells according to protocol described in Voo, et al. All human donor blood was obtained under informed consent, and used/disposed according to Astrazeneca's Human Biological Sample policy.

At day 8, TR1 cells were collected from culture, washed in PBS and stained for flow cytometry analysis to confirm a Treg marker phenotype. Cells were resuspended in PBS+2% FBS (Life Technologies) to a concentration of 0.5×10⁶ cells/0.1 mL and stained with a multicolor antibody panel consisting of CD3-APC-Cy7, CD25-BV711 (both obtained from Biolegend), CD4-FITC, FOXP3-PE, and CCR6-DL647 (all obtained from BD Biosciences) at suppliers recommended concentrations. Prior to cell surface staining, cells were treated with Zombie-UV (Biolegend) according to supplier's protocol to gate out dead cells from the analysis. Following this, live cells were stained for all cell surface markers for 15 min. on ice. Stained cells were then washed twice with PBS+2% FBS and fixed using BD cytofix/cytoperm kit (BD Biosciences) according to recommended protocol. Following the fixation step, cells were stained for intracellular FOXP3 for 15 min. on ice, followed by an additional 2 washes in PBS+2% FBS. Flow cytometry analysis was performed on a BD Fortessa and analyzed by FlowJo software. Multicolor compensation was performed using compensation beads (eBiosciences), stained and analyzed in parallel to the cells, and Full-minus-one (FMO) controls were set up for each color to determine positive/negative gating.

TR1 Chemotaxis Assay.

NCIH508 colon cancer cells were plated to ultra-low attachment plates (Corning) in serum-free defined media as described above to promote CSC-driven sphere growth. After 4 days of undisturbed culture, conditioned medium (CM) from the cells was collected, 0.2 μM filtered, and stored at 4° C. until further use. CCL20 concentration in the media was determined by ELISA as described above. This CSC CM was used within a week of harvesting for chemotaxis assays.

TR1 cells harvested at day 8 of culture were washed in PBS and resuspended to either PBS+2% FBS or serum free defined media (SCM) at 0.4×10⁶ cells/mL. Isolated CCL20 (R&D systems) was prepared at 100 nM concentration in PBS+2% FBS. Anti-CCL20 monoclonal antibody or isotype control antibody (both from R&D systems) was added to either isolated CCL20 or CSC CM at 667 nM final concentration and allowed to incubate for 30 min. at room temperature. Prepared treatments were transferred to the receiving wells of a transwell migration plate with 8 μM pores (Neuroprobe) at n=4 wells/treatment. TR1 cells were plated on top of the filter plate according to suppliers protocol at 50,000 cells/well in matched medium (PBS+2% FBS for isolated CCL20 treatments, and SCM for CSC CM treatments). Plates were allowed to incubate for 60 min. at 37° C., after which non-migrated cells were blotted off the filter plate, and the filter was gently washed with PBS. Plates were centrifuged at 200 rpm for 2 min. to collect migrated cells in the receiving wells. Numbers of migrated cells were determined by cell titer glo assay (Promega) according to suppliers protocol, whereby a standard curve of cell number vs. relative luminescence (RLU) was used to convert RLU to % of TR1 cells migrated. Statistical significance was determined by 2-tailed student's T-test between treatment and control, whereby n=4 replicates/treatment.

Beta-Arrestin and cAMP Pathway Assays.

Beta-arrestin recruitment was measured as a reporter for both human and mouse CCL20-CCR6 pathway activation using the PathHunter® eXpress CCR6 CHO-K1 β-Arrestin GPCR Assay (DiscoverX), whereby cells stably expressed mouse CCR6 for the mouse pathway assay and human CCR6 for the human pathway assay. Assays were run according to supplier's protocol with the adjustment that cells were stimulated with 6 nM recombinant human CCL20 (R&D Systems) or 1.2 nM recombinant mouse CCL20 (R&D systems) for 90 min at 37° C. Changes in cAMP levels were additionally measured in a separate assay as a reporter for human CCL20-CCR6 pathway activation using the LANCE cAMP assay kit (Perkin Elmer) according to supplier's protocol, using Ad293 cells stably transfected with human CCR6. In this assay, cells were stimulated with 4 μM forskolin (Sigma Aldrich) in combination with 1 nM recombinant CCL20 for 60 min at room temperature. In both assays, pathway neutralization was measured following treatment with CCL20 IgG or isotype control IgG (both R&D systems).

In-Vivo Syngeneic Mouse Models.

4T1 mouse breast carcinoma was xenografted subcutaneously into the hind flank of 4-6 week old female Balb/c mice (BALB/cAnNHsd, Envigo). When tumors reached approximately 50-100 mm³, mice began receiving treatment with either an anti-mouse CCL20 monoclonal antibody (clone 114908) or an isotype control antibody (both R&D systems), dosed at 10 mg per kg i.p. twice weekly for 4 consecutive doses. Mice were humanely euthanized at 24 hours following the final dose and spleens and tumors were collected for analysis. Both spleen and tumor tissue was digested to single cell suspension and resulting cells were cryopreserved in Cryostor CS5 (Sigma-Aldrich) prior to flow cytometric analysis of immune cells.

Mouse Immune Cell Flowcytometric Analysis.

Cryopreserved spleen and tumor cells from mice treated with either isotype or anti-mouse CCL20 monoclonal antibody were thawed at 37° C., washed, and stained for mouse immune cell analysis using the following antibody panel: anti cd45-BV21, anti cd4-BV711, anti cd11b-BV510 (all obtained from BD Biosciences), anti cd3-FITC, anti cd8a-APC-Cy7, anti foxp3-PerCP-Cy5.5, anti rorγt-PE (all obtained from eBiosciences), anti cd11c-BV605, anti-cd83-PE-Cy7, anti ly6g-BV785 (all obtained from Biolegend). Live cells were first blocked with 4% mouse serum (Jackson Immunoresearch), subsequently stained for live/dead cells using Zombie-UV (Biolegend), then stained for extracellular antigens (all targets except for foxp3 and rorγt). Following this, cells were washed and fixed and permeabilized using a Cytofix/Cytoperm kit (BD Biosciences) prior to staining the cells with antibodies against intracellular antigens foxp3 and rorγt. Flow cytometry was run on a BD Fortessa equipped with 355 nm, 405 nm, 488 nm, 532 nm, and 640 nm laser excitation, and analyzed using flowjo V10 software. Statistical significance was determined by 2-tailed student's T-test between treatment and control, whereby n=5 mice/treatment/tissue type. Data points that fell beyond 3× standard error of the mean were identified as outliers and removed from analysis.

Human CD4+ T Cell Chemotaxis Assay.

CD4+ T cells were isolated from healthy human donor blood by negative selection using the Rosettesep CD4+ T cell enrichment kit (Stemcell Technologies). Cells were washed in PBS and resuspended to PBS+2% FBS. Isolated CCL20 (R&D systems) was prepared at 10 nM concentration in PBS+2% FBS. Anti-CCL20 monoclonal antibody or isotype control antibody (both from R&D Systems) was added to isolated CCL20 at 30 nM final concentration and allowed to incubate for 30 min at room temperature. Prepared treatments were transferred to the receiving wells of a transwell migration plate with 5 μM pores (Corning) at n=3 wells/treatment, and CD4+ T cells were loaded into the insert over a polycarbonate filter at 50,000 cells/well in matched medium (PBS+2% FBS), and plates were allowed to incubate for 3 hours at 37° C. Numbers of migrated cells were determined by cell titer glo assay (Promega) according to supplier's protocol, whereby a standard curve of cell number vs. relative luminescence (RLU) was used to convert RLU to % of CD4+ T cells migrated. Statistical significance was determined by 2-tailed student's T-test between treatment and control, whereby n=3 replicates/treatment/donor.

### Example 1: Expression of CCL20 in Cancer Stem Cells in Culture

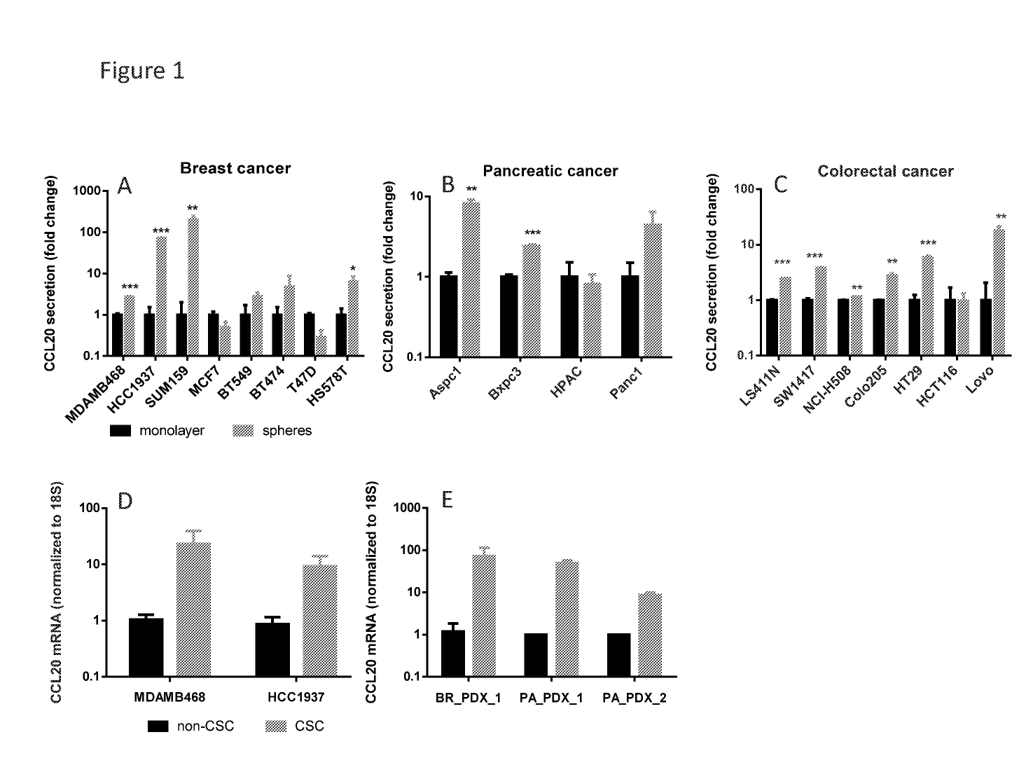

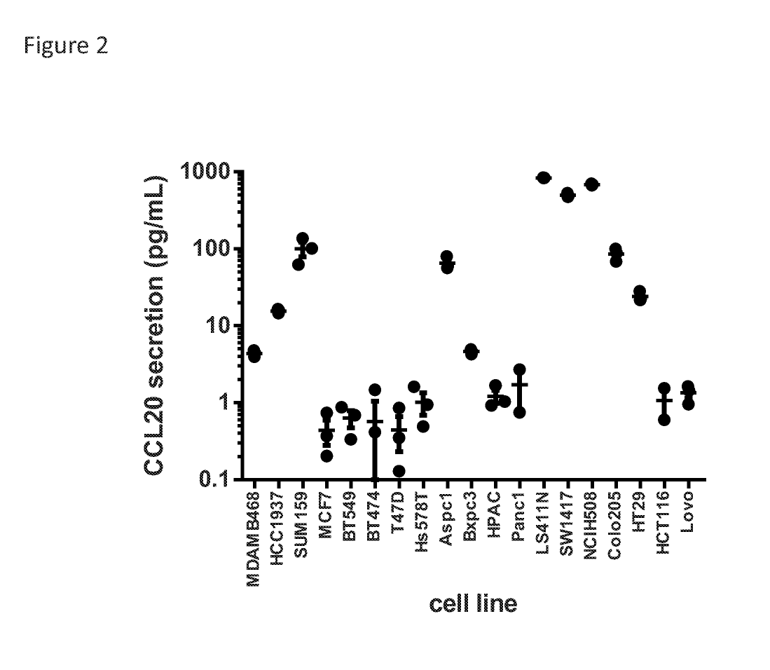

As discussed above, although CCL20 is known as a chemo-attractant for the recruitment of immune cells, the results discussed herein demonstrate that CCL20 is a factor produced by CSCs in cancer cell lines, some of which are exemplified herein, including breast, pancreatic, and colorectal cancers. In tissue culture, the majority of cell lines are found to profoundly increase secretion of CCL20 when cultured under CSC-enriching sphere conditions relative to monolayer culture conditions (FIGS. 1A-1C). As FIG. 1 shows, sphere culture conditions resulted in a significant increase in CCL20 secretion when compared to monolayer culture in many of the breast cancer lines (FIG. 1A), pancreatic cancer lines (FIG. 1B), and colorectal cancer lines (FIG. 1C) that were tested. Typical levels of CCL20 that were secreted by cells under sphere culture conditions ranged from about 1 to about 1000 pg/mL, whereby cell lines that lacked a CCL20 increase in sphere culture generally had low or undetectable protein levels of at or below 1 pg/mL (FIG. 2).

To confirm that the sphere-associated increase in CCL20 levels are attributable to CSCs, fluorescence activated cell sorting (FACS) was used to isolate CSCs and non-CSCs from breast and pancreatic cancer lines, in order to measure the levels of CCL20 mRNA in each cell population. Isolated CD44^{high/+} CD24^{low/-} CSCs from two breast cell lines (MDAMB468 and HCC1937) have dramatically elevated CCL20 mRNA levels compared with CD44^{low/-} CD24^{low/-} non-CSCs isolated from the same lines (FIG. 1D). CSCs from MDAMB468 and HCC1937 cells produce 23-fold and 10-fold higher CCL20 transcript than non-CSCs, respectively (RE for CSCs vs. non-CSCs=23.3±16 vs. 1.0±0.2 in MDAMB468 and 9.3±4.6 vs. 0.9±0.3 in HCC1937). To assure clinical relevance of this observation, CCL20 transcript levels were also measured in FACS isolated CSCs versus non-CSCs from primary patient derived tumor xenografts (PDX). Tumors from two pancreatic cancer (PA_PDX_1 and PA_PDX_2) and one breast cancer (BR_PDX_1) PDX model were similarly FACS sorted to isolate Epcam⁺ CD44⁺ CD24⁺ CSCs along with Epcam⁻ CD44⁻ CD24⁻ non-CSCs for pancreatic cancer, and Epcam⁺ CD44⁺ CD24⁻ CSCs along with Epcam⁺ CD44⁻ CD24⁻ non-CSCs for breast cancer. As shown in FIG. 1E, all three patient xenograft models showed a significant increase in CCL20 mRNA levels in isolated CSCs over non-CSCs, with relative expression for CSCs vs. non-CSCs=71.8±41.6 vs. 1.2±0.6 (for BR_PDX_1), 48.8±8.6 vs. 1.0±0.0 (for PA_PDX_1), and 8.6±1.3 vs. 1.0±0.0 (for PA_PDX_2).

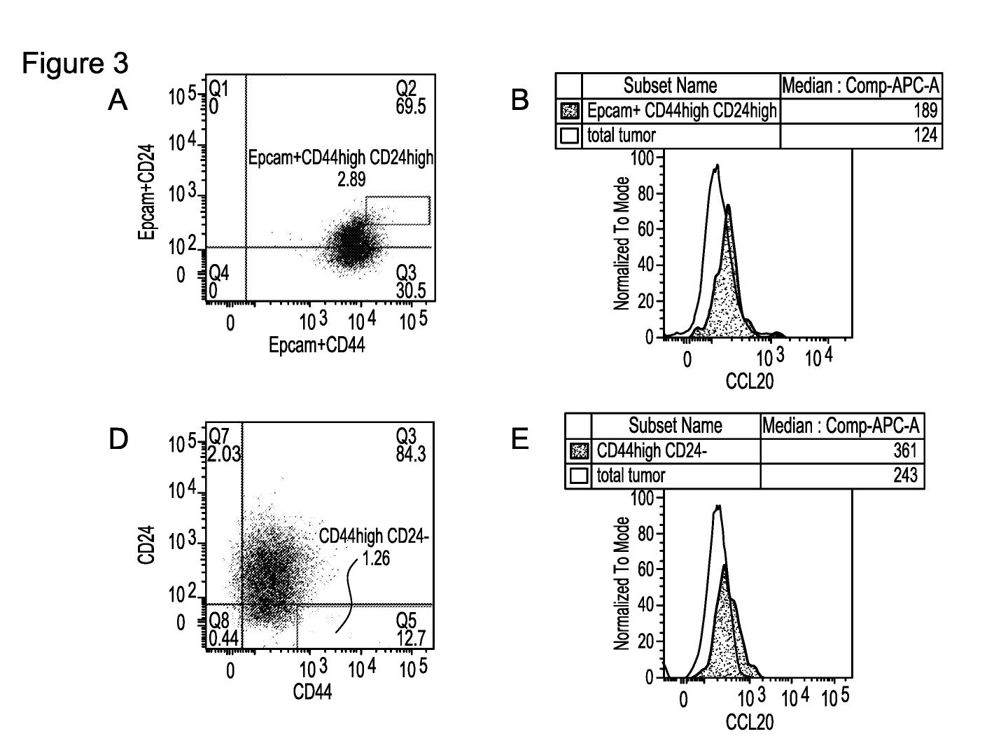

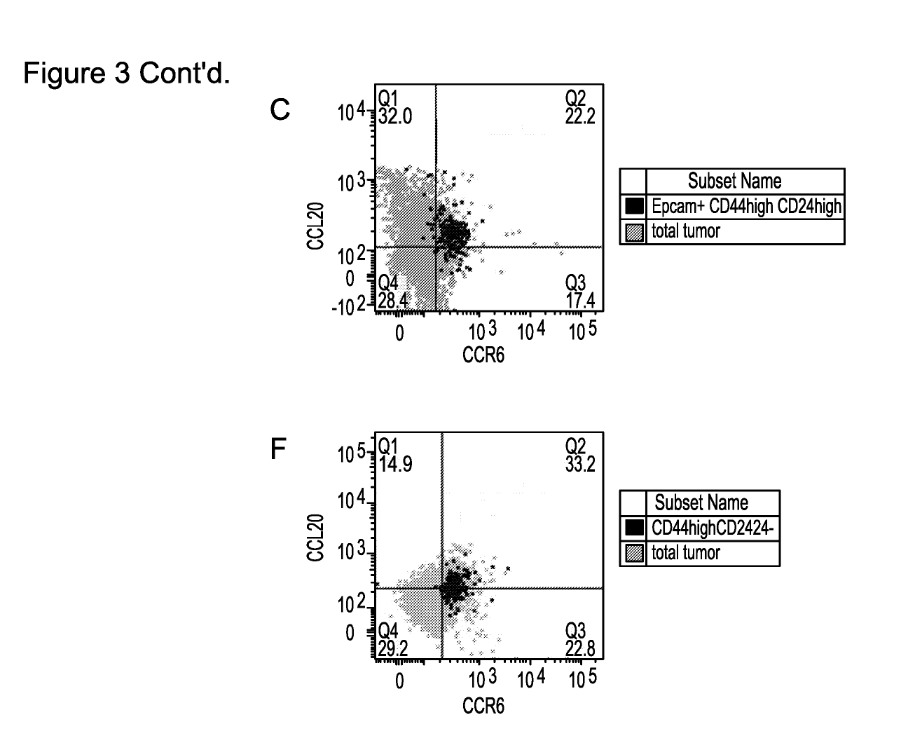

Next, we confirmed by flow cytometry that CSCs indeed are marked by elevated CCL20 levels compared to the rest of the tumor cell population. While CSCs are commonly identified as Epcam⁺CD44⁺CD24⁺ in pancreatic cancer and CD44⁺CD24⁻ in breast cancer, we find that the stem cell phenotype, as determined by tumorigenicity, sphere formation, and stemness gene expression often tracks better in monolayer cell line models with high expression of CD44 and/or CD24 (thus Epcam⁺CD44^{high}CD24^{high} in monolayer cultured pancreatic cancer and CD44^{high}CD24⁻ in monolayer cultured breast cancer). By this identification, we found that CSCs from the Bxpc3 pancreatic model (FIG. 3A) and from the HCC1937 breast cancer model (FIG. 3D) both showed a 1.5-fold increased level of CCL20 median fluorescence intensity (MFI) over the total tumor cell population. Bxpc3 CSCs (FIG. 3B) exhibited a shift in MFI of 189, while the total tumor cell population averaged a CCL20 MFI of 124. Similarly, HCC1937 CSCs (FIG. 3E) also showed an increased shift in CCL20 MFI from 243 in the total tumor cells to 361 specifically for the CD44^{high}CD24⁻ CSCs. This data corroborates the findings and results depicted in FIG. 1 which show that elevated CCL20 levels associate predominantly with CSC-enriched spheres and isolated CSCs. CSCs gating was determined by full-minus one staining as depicted in FIG. 13.

### Example 2: Association of CCR6 and Co-Localization with CCL20 in Cancer Stem Cells

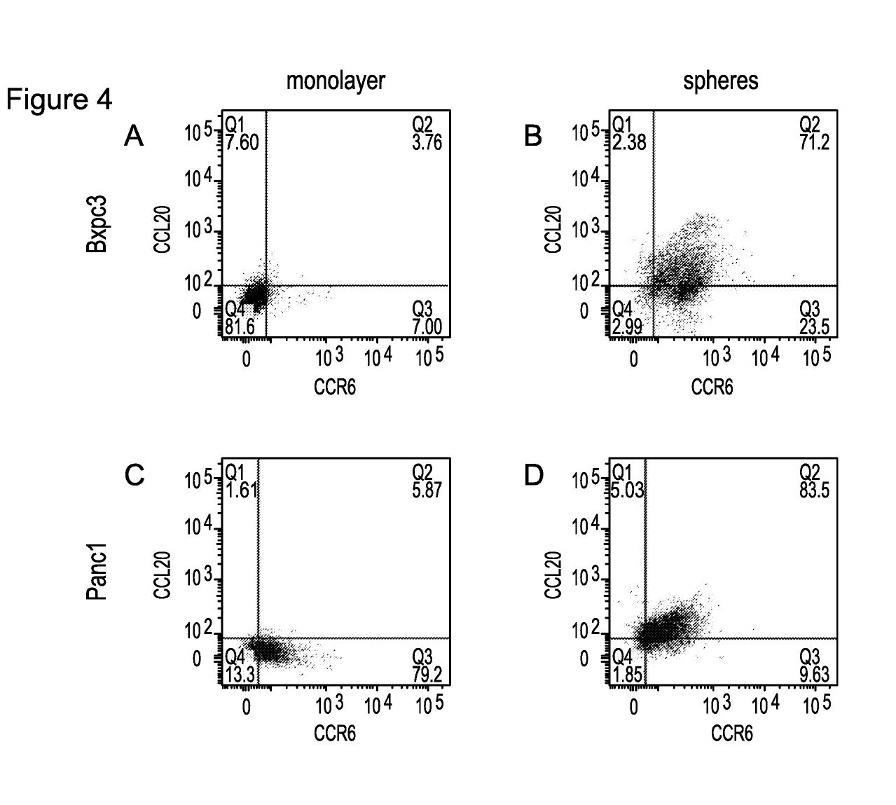

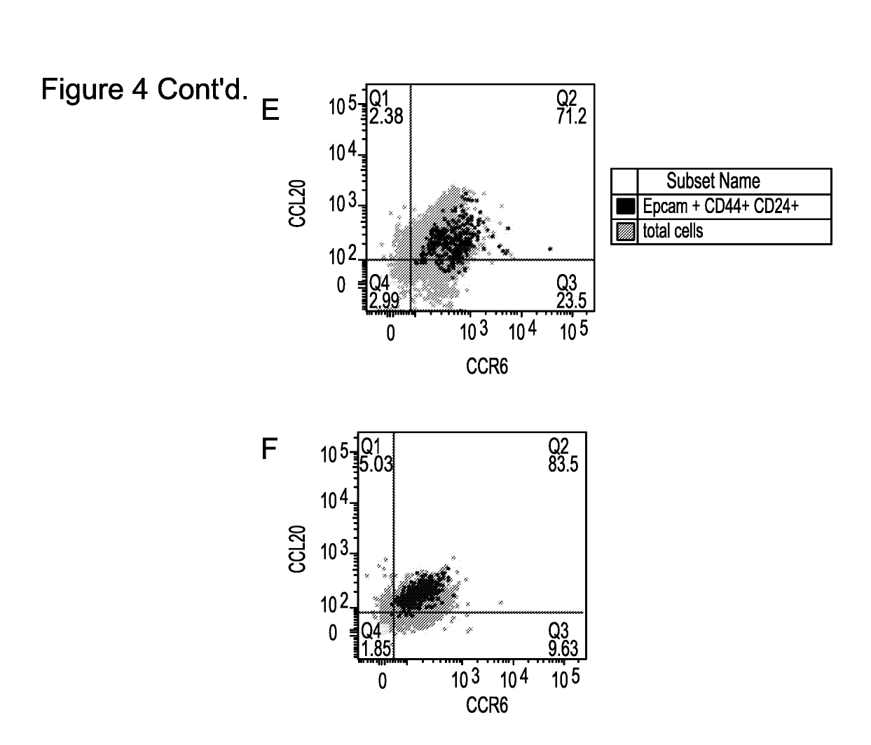

Interestingly, when we overlaid the CSC populations with CCL20/CCR6 staining for the tumor cells, we found that the majority of CSCs from both Bxpc3 and HCC1937 lines appear to co-localize with cells that are double positive for both the ligand and the receptor, ie. CCL20⁺CCR6⁺ (FIGS. 3C & 3E). Strikingly, we discovered that sphere culture dramatically increases this double positive CCL20⁺CCR6⁺ population over monolayer culture. FIG. 4 illustrates this effect in Bxpc3 and Panc1 pancreatic cancer models. When cultured in standard monolayer, a small fraction of cells in both models are identified as CCL20⁺CCR6⁺ (FIGS. 4A & 4C, and FIG. 13 for gating controls). However, when cultured as spheres, the majority of cells in both models stain positive for both CCL20 and CCR6 (FIGS. 4B & 4D). In the Bxpc3 model, this CCL20⁺CCR6⁺ population increases from 3.76% (FIG. 4A) as monolayer to 71.2% (FIG. 4B) in spheres. Similarly, Panc1 cells cultured as monolayer have 5.87% CCL20⁺CCR6⁺ cells (FIG. 4C), a population that is boosted to 83.5% in sphere culture (FIG. 4D). Interestingly, when we gate on the Epcam⁺CD44⁺CD24⁺ cells in these spheres, they again overlay almost completely with the CCL20⁺CCR6⁺ population in Bxpc3 (FIG. 4E) and Panc1 (FIG. 4F) spheres. These observations suggested that CSCs may have an autocrine dependence on the CCL20-CCR6 axis.

### Example 3: Correlation of CCL20 Levels and Cancer Stem Cell Activity

Following the confirmation that CCL20 is indeed overexpressed by and associated with the CSC population, we sought to confirm the phenotypic relevance of this molecule to CSC activity. FIG. 5 illustrates how exogenous addition of purified CCL20 boosts sphere growth, which serves as a model for CSC-driven tumorigenicty. The addition of recombinant human CCL20 (rhCCL20) to the CSC culture significantly stimulated sphere growth at day 4 in a dose-dependent manner in three models of cancer, Aspc1 (FIG. 5A), Bxpc3 (FIG. 5B), and SUM159 (FIG. 5C). While the addition of CCL20 causes a dose-dependent increase in sphere growth, the ligand shows no effect on stimulating growth of cells in standard monolayer culture (FIGS. 5A-5C). This observation tends to suggest that the effect of CCL20 is directed specifically toward CSCs. The specificity of this effect was confirmed in FIG. 5D, which illustrates that a 50 pg/mL dose of CCL20 obtained from two separate commercial sources (CCL20 A and CCL20 B) stimulates sphere growth of BT549 breast cancer cells to the same extent. For example, CCL20 A stimulated growth to 333.3±64.9% compared with 100.0±16.7% for basal cells, while CCL20 B stimulated growth to 300.0±54.6% over basal (p<0.01 and p<0.05, respectively, when compared with basal sphere growth). Moreover, this stimulatory effect can be neutralized or nullified upon addition of anti-CCL20 monoclonal antibody (15 μg/mL), whereby despite the presence of the same 50 pg/mL CCL20 A or CCL20 B, sphere growth was maximally 154±16.9% for CCL20 A+anti-CCL20 antibody and 125.0±16.1% for CCL20 B+anti-CCL20 antibody (see, FIG. 5D; p<0.05 compared to CCL20-treated spheres without anti-CCL20 antibody).

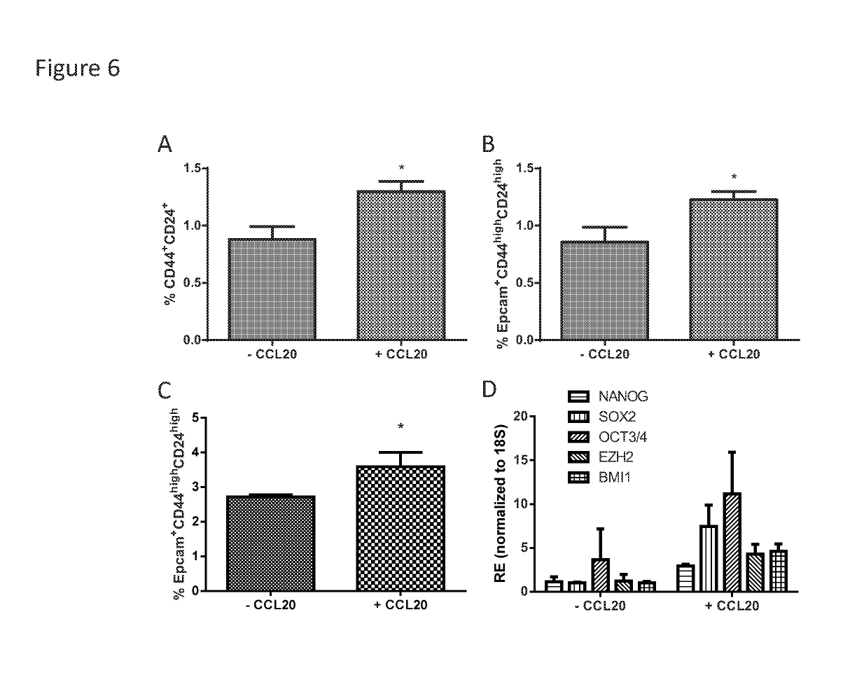

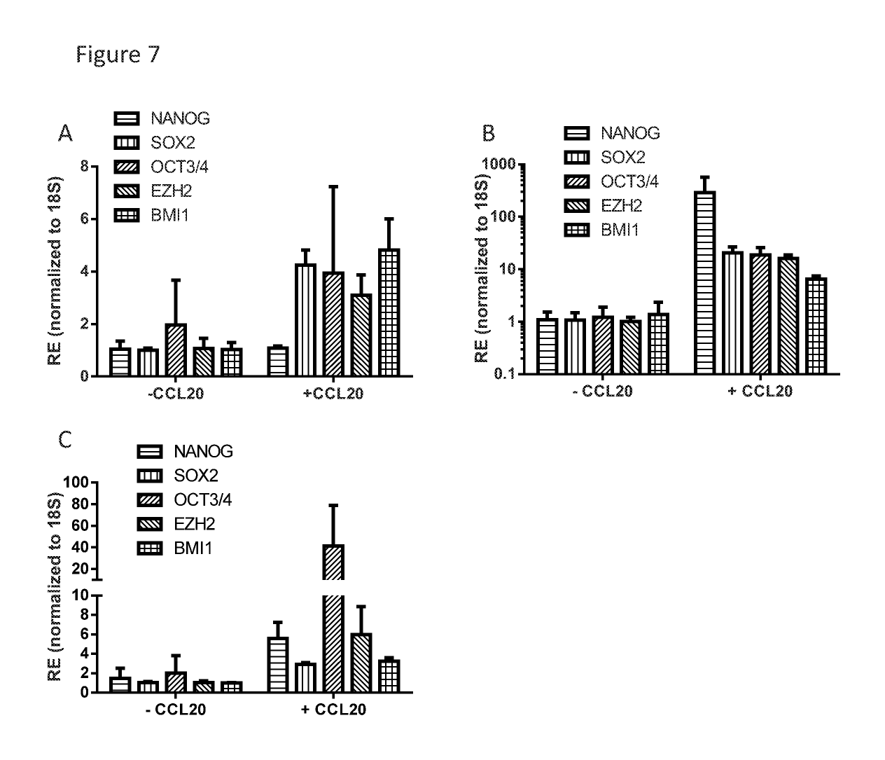

Overall the specificity of this response confirms that CCL20 indeed mediates phenotypic CSC activity. However, since sphere growth is an indirect measure of CSC activity, we sought to confirm that CCL20, as an isolated factor, increases CSCs directly. In brief, spheres treated with or without 200 pg/mL CCL20 for 4 days were subjected to flow cytometry analysis to directly measure CCL20-driven increases in the CSC population. FIG. 6 demonstrates that CCL20 treatment significantly increases the percentage of CD44⁺CD24⁻ breast CSCs from 0.9±0.1% (without CCL20) to 1.3±0.1% (with CCL20) in MDAMB468 cells (p<0.05) (FIG. 6A). Similarly, Bxpc3 (FIG. 6B) and Aspc1 (FIG. 6C) pancreatic cancer cells show significant increases in their Epcam⁺CD44^{high}CD24^{high} CSC frequencies following CCL20 stimulation, from 0.9±0.1% to 1.2±0.1% in Bxpc3 and from 2.7±0.1% to 3.6±0.4% in Aspc1 (p<0.05 in both cell types). In addition to sphere growth and flow cytometry we, and others, have found that a panel of five stemness-related genes also tracks well with the CSC phenotype. Expression of the transcription factors NANOG, SOX2, and OCT3/4, which are known to be associated with pluripotency, increases and decreases with corresponding increases and decreases in the CSC population. Transcript expression of polycomb proteins EZH2 and BMI1 both relate similarly well with changes in the CSC population. FIG. 6D shows that addition of 200 pg/mL CCL20 to Bxpc3 spheres indeed increases expression of all 5 stemness-related genes after 4 days of treatment. Similar results were found in Hs578T, SUM159, and Panc1 cell lines (FIG. 7). Together with the effects seen in sphere growth and the increases measured in the percentage of cells bearing CSC surface markers, the data indeed suggests that the ligand CCL20 can directly promote CSC activity in the cell lines tested.

### Example 4: Effect of CCL20 in Cancer Stem Cells is Mediated by its Receptor, CCR6

While the results of the experiments discussed in Examples 1-3 confirmed that CCL20 directly affected CSC activity, it remained to be determined whether CCL20 indeed signaled through its known receptor, CCR6, to exert this activity. The data depicted in FIG. 5 showed that addition of purified CCL20 stimulated sphere growth. From this an experiment was designed to assess whether the effect of CCL20 was mediated through signaling through its receptor CCR6, reasoning that if CCL20 exerted its function through CCR6 signaling, knockdown of CCR6 would prevent CCL20-stimulated sphere growth.

In FIG. 8A, we show that recombinant human CCL20 (rhCCL20) loses the ability to stimulate sphere growth in Lovo colorectal cancer cells following knockdown of CCR6 with 20 nM siRNA, while cells transfected with non-target control siRNA (20 nM) retained their growth response to CCL20. To confirm that CCR6 was a necessary component to this CCL20 axis on CSCs, we separated CCR6⁺ from CCR6⁻ cells in the NCIH508 cell line, and stimulated both fractions with 100 pg/mL CCL20 for 48 hours and subsequently evaluated stemness-related gene expression. FIG. 8B shows that CCR6⁺ cells responded with about a 2-fold increased expression of NANOG, BMI1, OCT3/4, and SOX2 after 48 hours of stimulation with 100 pg/mL, when compared with CCR6⁻ cells. These results demonstrate that CSC-related activity in response to CCL20 stimulation is dependent on the presence of CCR6 as the receptor. To further support this finding, we observed that inhibition of this CCL20-CCR6 axis on the tumor cells, either by loss of CCL20 or by loss of CCR6, resulted in similar outcome of sphere growth (FIGS. 8C & 8D). RNAi-mediated knockdown of either CCL20 or CCR6 reduced sphere growth to a similar extent in NCIH508 cells, namely 59.6±2.9% or 70.3±2.2% compared to non-target control siRNA (100.0±2.0%, p<0.001) (FIG. 8C). The same observation was made in Hs578T cells where RNAi-mediated knockdown reduced sphere growth to 55.4±3.9% with CCL20 siRNA and 21.8±1.8% with CCR6 siRNA, compared to 100.0±7.0% growth with control siRNA (p<0.001) (FIG. 8D). Altogether the results confirm that the CCL20-CCR6 axis is responsible for promoting the effects of CCL20 on CSCs.

### Example 5: Neutralizing CCL20 Inhibits Cancer Stem Cell Activity

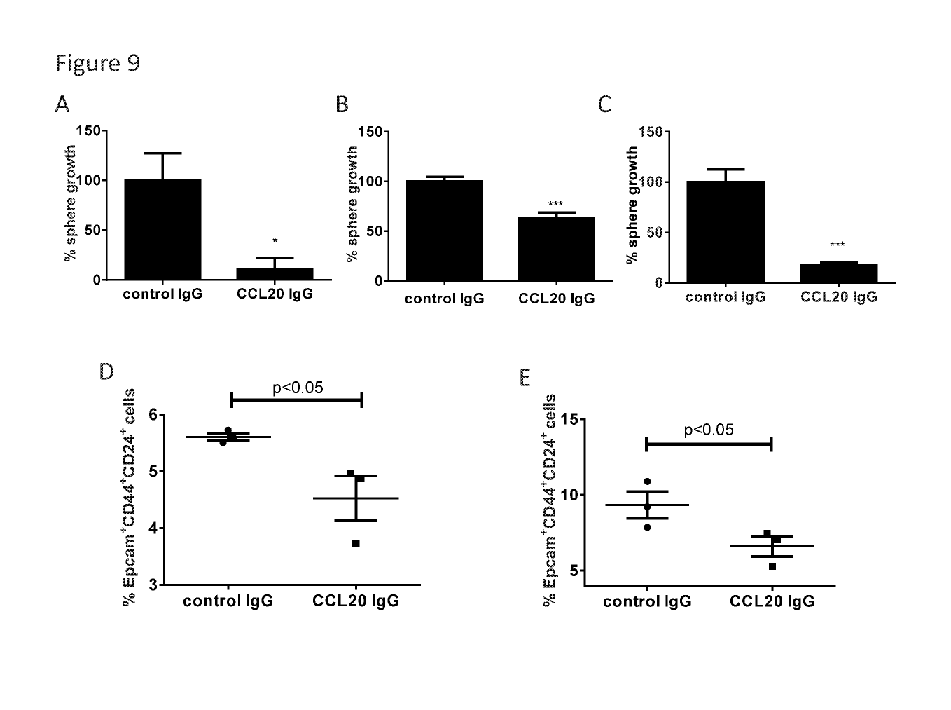

As the above Examples validate, CCL20 is a clear mediator of CSC activity. This example assessed whether neutralization of this target could then in turn inhibit CSCs. To test this, a commercially available monoclonal IgG antibody against CCL20 that had reported neutralizing potential was used in a series of experiments. A single dose administration of 10 μg/mL CCL20 IgG significantly reduced sphere formation at 4 days following treatment in models of all three indications tested, compared with the same 10 μg/mL dose of isotype control IgG (FIG. 9). Growth of Hs578T breast cancer spheres was reduced to a mere 11.1±11.1% compared to 100.0±27.2% sphere growth with control IgG (p<0.05, FIG. 9A). Similarly, growth of NCIH508 colorectal cancer spheres was reduced to 62.8±5.8% vs. 100.0±4.8% with control IgG (p<0.001, FIG. 9B), while growth of Panc1 pancreatic cancer spheres reduced to 18.1±2.1% down from 100.0±13.0% with control IgG treatment (p<0.001, FIG. 9C). Gating controls for CSC identification are illustrated in FIG. 15.

The effect of CCL20 inhibition on CSC reduction was confirmed by flow cytometry, where a similar 4 day treatment of Bxpc3 spheres with a single dose administration of 10 μg/mL CCL20 IgG decreased the percentage of Epcam⁺CD44⁺CD24⁺ CSCs to 4.5±0.4%, down from 5.6±0.1% with 10 μg/mL control IgG treatment (p<0.05, FIG. 9D). The frequency of Panc1 pancreatic CSCs was reduced in spheres from 9.3±0.9 with control IgG treatment to 6.6±0.7% after CCL20 IgG (p<0.05, FIG. 9E). Altogether, these results confirm that neutralization of CCL20 with a monoclonal antibody can indeed be used as a potential therapeutic to reduce the CSC population, revealing a promising opportunity for CCL20 as a target for anti-CSC therapies.

### Example 6: T_{reg} Mobility is Mediated by CCL20 Produced by CSCs

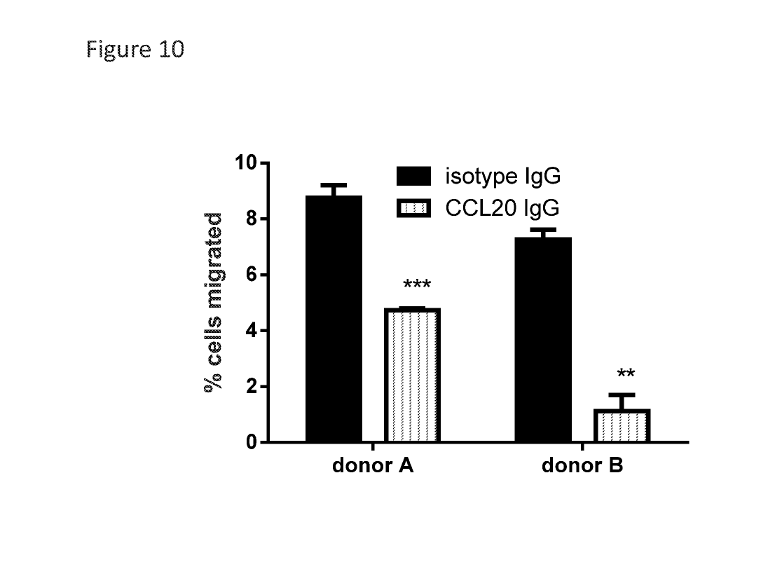

CCL20 is known to direct chemotactic migration of CCR6+ immune cells, predominantly in the B cell, T cell, and dendritic cell subsets. As a positive control, we confirmed the ability of an anti-CCL20 IgG to block chemotactic migration of a known cell type, namely CD4+ T cells. FIG. 10 confirms that anti-CCL20 IgG, dosed at 30 nM, can significantly inhibit chemotactic migration of CD4+ T cells isolated from peripheral blood from 2 healthy human donors.

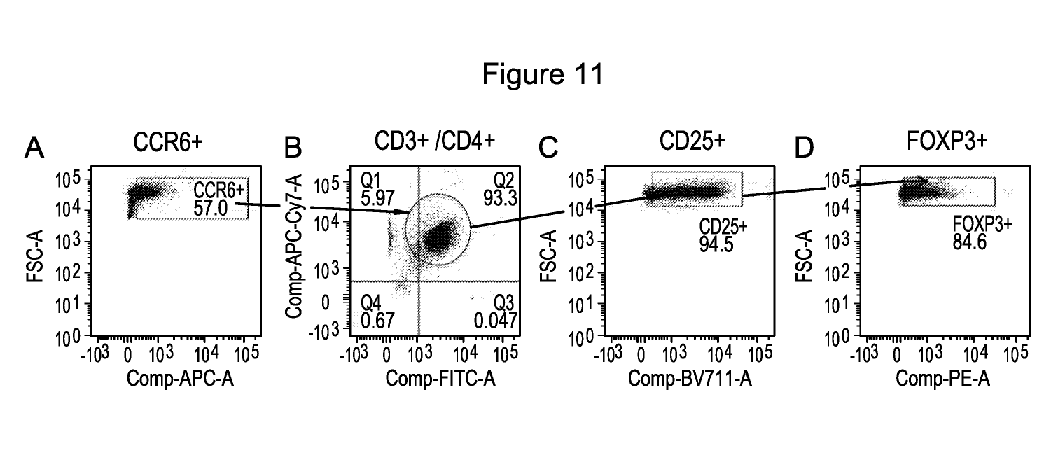

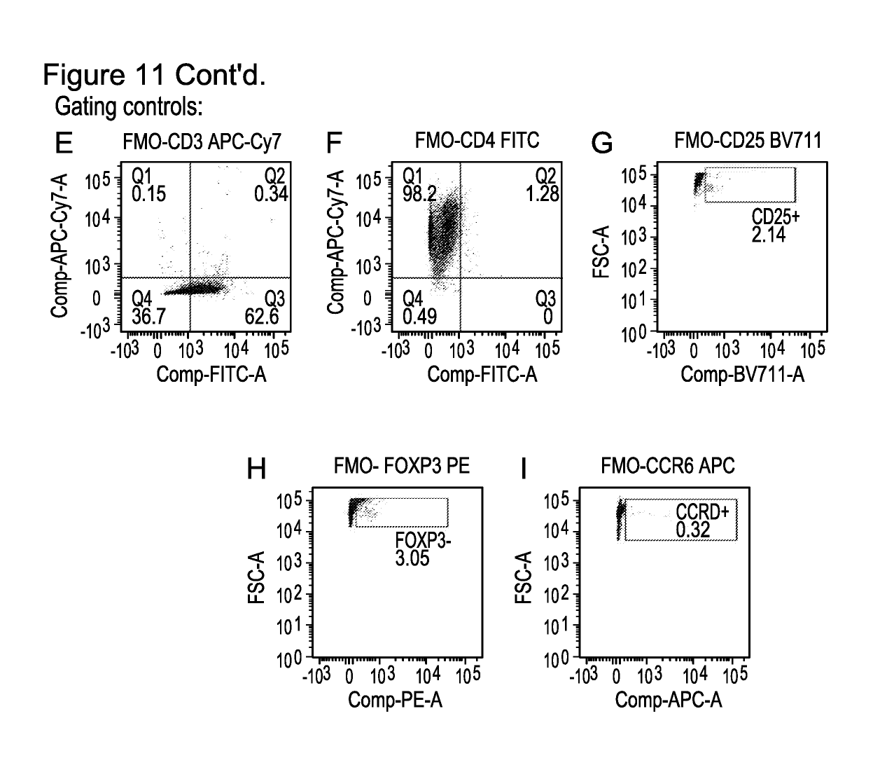

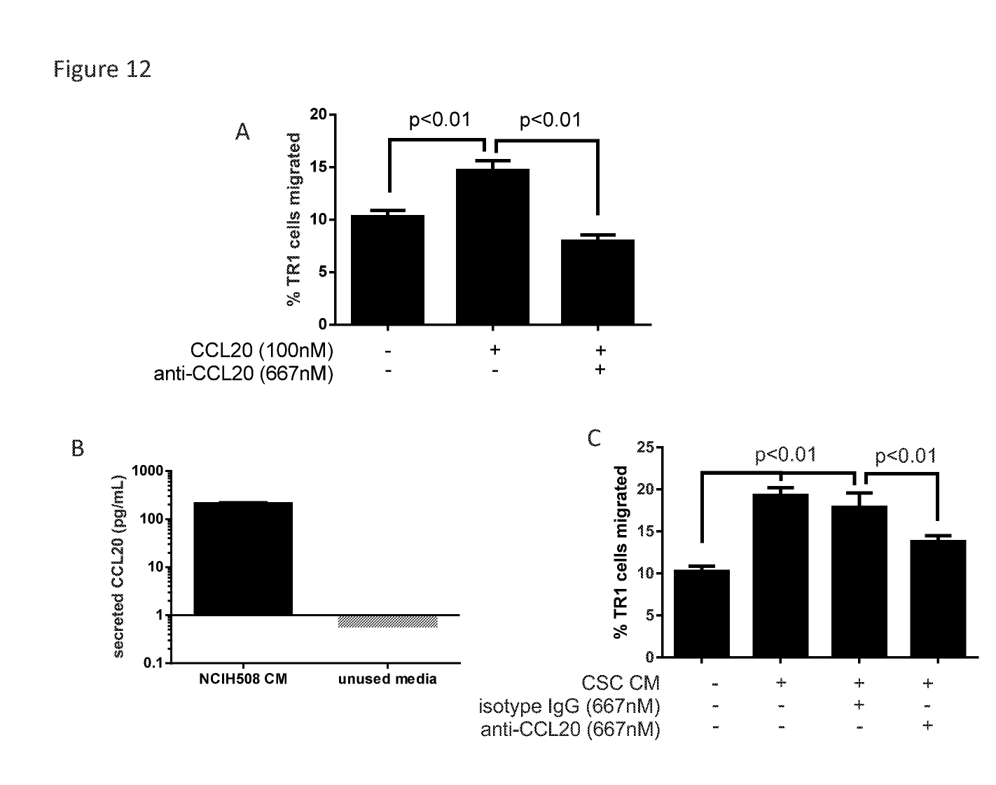

To test whether CCL20 produced by CSCs can influence migration of T_{regs}, we skewed human naïve CD4+ T cells towards type-1 regulatory T cells (TR1) in culture as described above in the materials and methods. Flow cytometry analysis of the cells confirmed that TR1 cells resemble T_{regs} by surface phenotype, as they are predominantly CD3+/CD4+/CD25+/FOXP3+ T cells, and that these TR1 cells expressed CCR6 (FIG. 11). A transwell migration assay revealed that these TR1 cells could selectively migrate towards isolated CCL20, whereby 14.7±0.9% of cells migrated towards CCL20, compared with 10.2±0.6% of cells that non-specifically migrated (p<0.01, FIG. 12A). Addition of the anti-CCL20 antibody at 6-fold molar excess was able to block this migration, seen by a reduction of cell migration to 7.9±0.6% (p<0.01 compared with 100 nM CCL20 treatment, FIG. 12A).

Next, we asked whether TR1 cells could migrate towards CCL20 produced by CSCs. We generated CSC-conditioned medium from NCIH508 colon cancer cells cultured under CSC-driven sphere forming conditions for 4 days, which was found to contain CCL20 at an average concentration of 211 pg/mL (=26.4 pM) as determined by ELISA (FIG. 12B). TR1 cells (19.3±0.9%) were able to migrate towards CSC CM compared with 10.3±0.6% of cells that non-specifically migrated (p<0.01, FIG. 12C). The migration was in fact directed towards CCL20 in the conditioned medium since addition of the neutralizing anti-CCL20 antibody significantly reduced migration back down to 13.8±0.7% (p<0.01 compared with CSC CM or CSC CM+isotype control IgG, FIG. 12C). Addition of an isotype control IgG did not have any effect on reducing TR1 migration towards CSC CM (17.9±1.7% migration with isotype control IgG). These results confirm the hypothesis that type-1 regulatory T cells can migrate towards CCL20 produced by CSCs, and thereby suggests that neutralizing antibodies against CCL20 may reduce the infiltration of T_{regs} into the tumor environment.

### Example 7: CCL20 Neutralizing Antibodies Reduce Tumorigenesis and Immunosuppression In Vivo

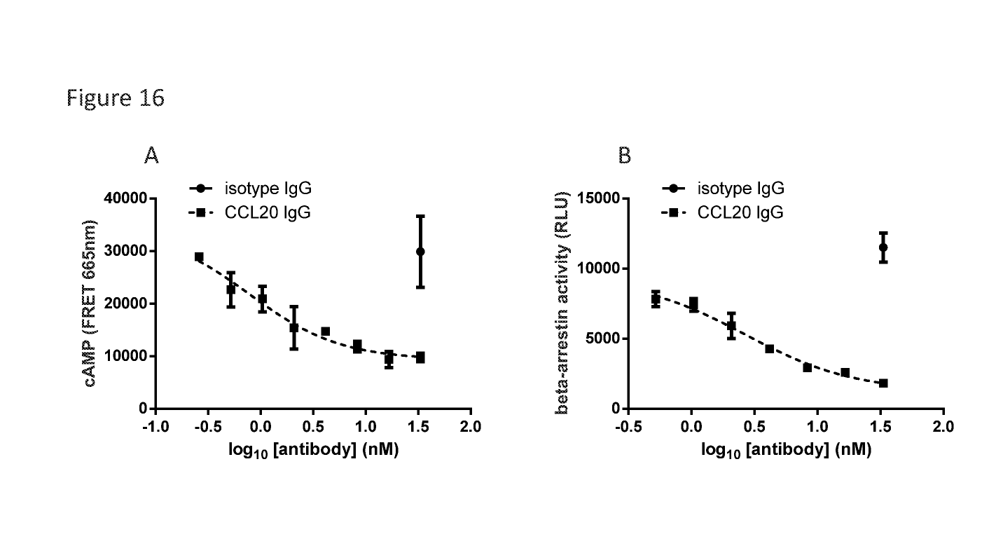

Once CCL20 was established as a clear mediator of CSC activity, we investigated whether neutralization of this target could then in turn inhibit CSCs. To test this, we used a commercially available neutralizing human monoclonal antibody against CCL20 (clone 67310; R&D Systems). Ability of this antibody to inhibit CCL20-stimulated CCR6 pathway activity in both a cAMP assay and a beta-arrestin translocation assay (FIGS. 16A and 16B, respectively) confirmed the neutralization potential. IC₅₀ was measured at 0.8 nM in a cAMP assay when cells were stimulated with 1 nM CCL20, while IC₅₀ was measured at 2.7 nM when cells were stimulated with 6 nM CCL20.

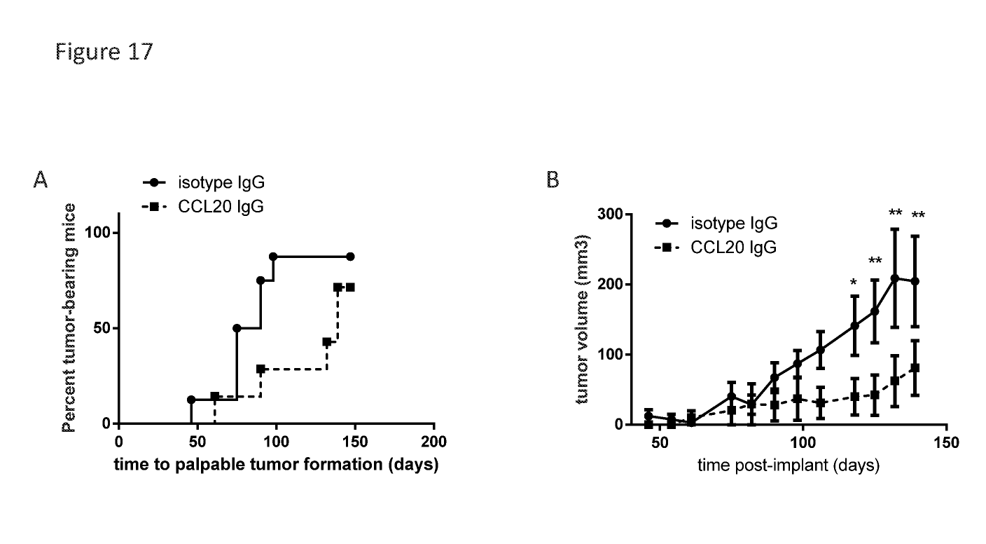

The reduction of CSC content through CCL20 neutralization in vitro translated into a delay in tumor take rate and tumor growth rate. Median time to tumor take of Panc1 tumors (% tumor-bearing animals) was delayed by 56.5 days when Panc1 tumors were pre-treated with 10 ug/mL CCL20 IgG compared to Panc1 tumors that were pre-treated with 10 ug/mL isotype IgG for 4 days in-vitro prior to tumor inoculation (FIG. 17A). Tumor take in this instance was measured from the first instance of palpable tumor formation. Average tumor volume for CCL20 IgG treated tumors was also significantly smaller than isotype IgG treated tumors over the course of study (FIG. 17B).

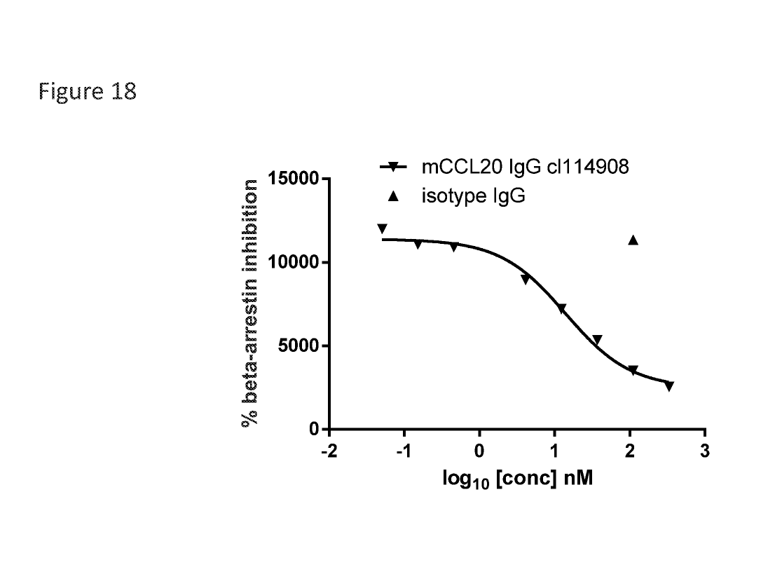

To evaluate the role of CCL20 neutralization in immune-competent syngeneic mouse models, we identified a commercially available anti-mouse CCL20 monoclonal antibody (clone 114908; R&D Systems) with sufficient blocking/neutralization potential to act as a surrogate for the anti-human CCL20 monoclonal antibody. The anti-mouse CCL20 IgG antibody was able to inhibit the mouse CCL20-mouse CCR6 pathway as measured by a beta-arrestin translocation assay with an IC₅₀ of 14.0 nM, when cells were stimulated with 1.2 nM mouse CCL20 (FIG. 18). However, potency of the mouse CCL20 IgG is noticeably reduced compared with the human CCL20 IgG (described in 0174) when the ratio of CCL20 to antibody IC₅₀ value is compared between human and mouse.

Syngeneic tumor models were used to evaluate the effect of in vivo CCL20 neutralization on peripheral and tumor-infiltrating immune cells. The 4T1 mouse breast carcinoma cell line is known to naturally express CCL20 and therefore chosen for this study. Immune-competent mice were xenografted subcutaneously with HLA-matched 4T1 cells, and dosed with anti-mouse CCL20 IgG or matched isotype control IgG at 10 mg per kg twice weekly for 4 consecutive doses once tumors had reached approximately 50-100 mm³. Post-mortem immunophenotyping was performed on spleen and tumor tissue harvested 24 hours following the 4^{th} dose.

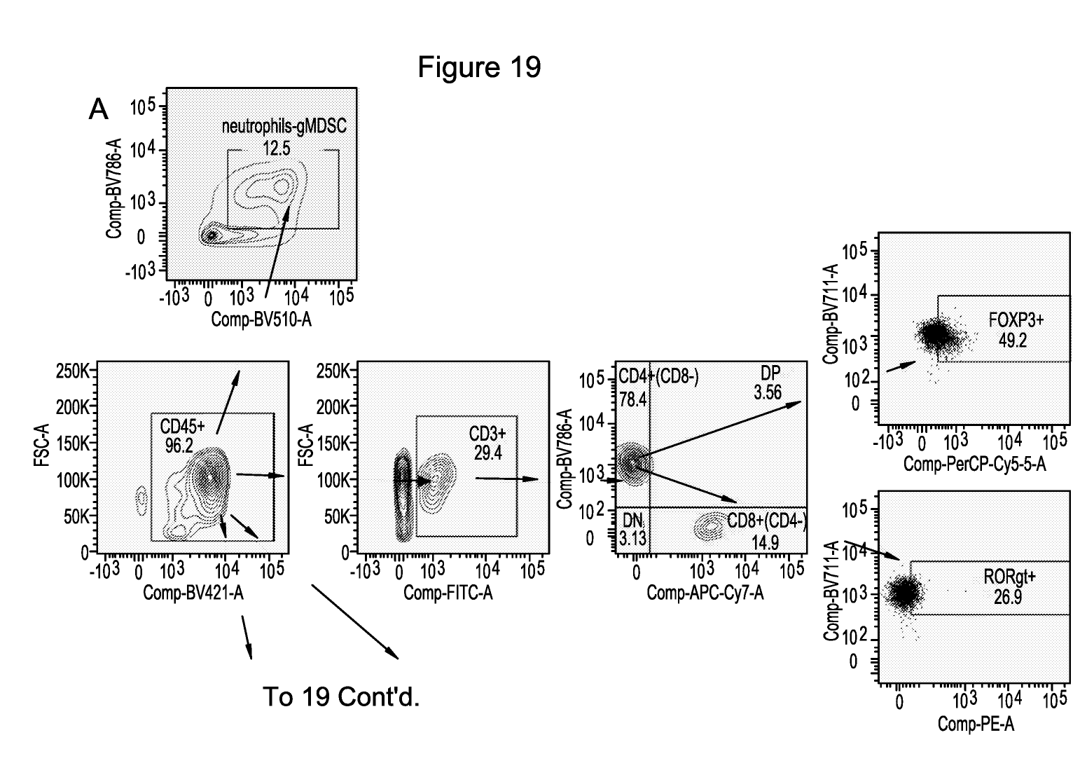

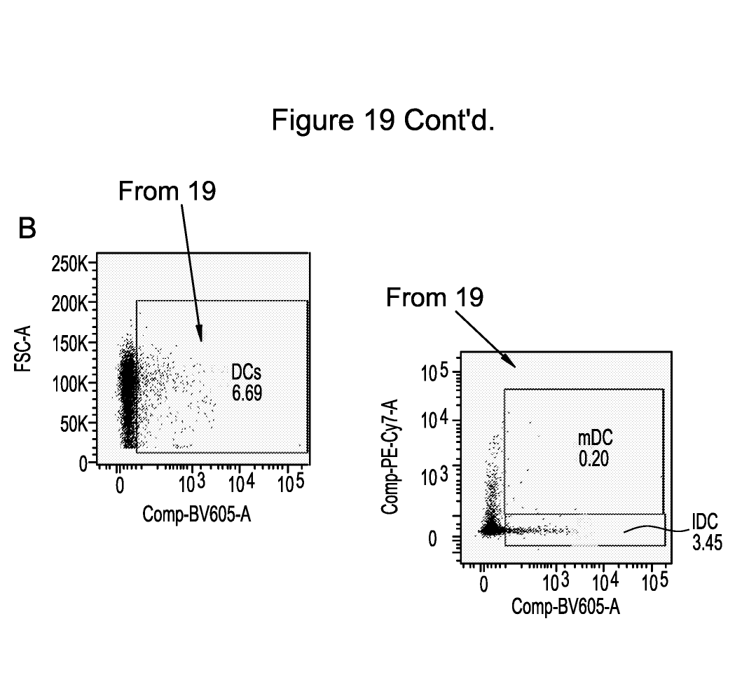

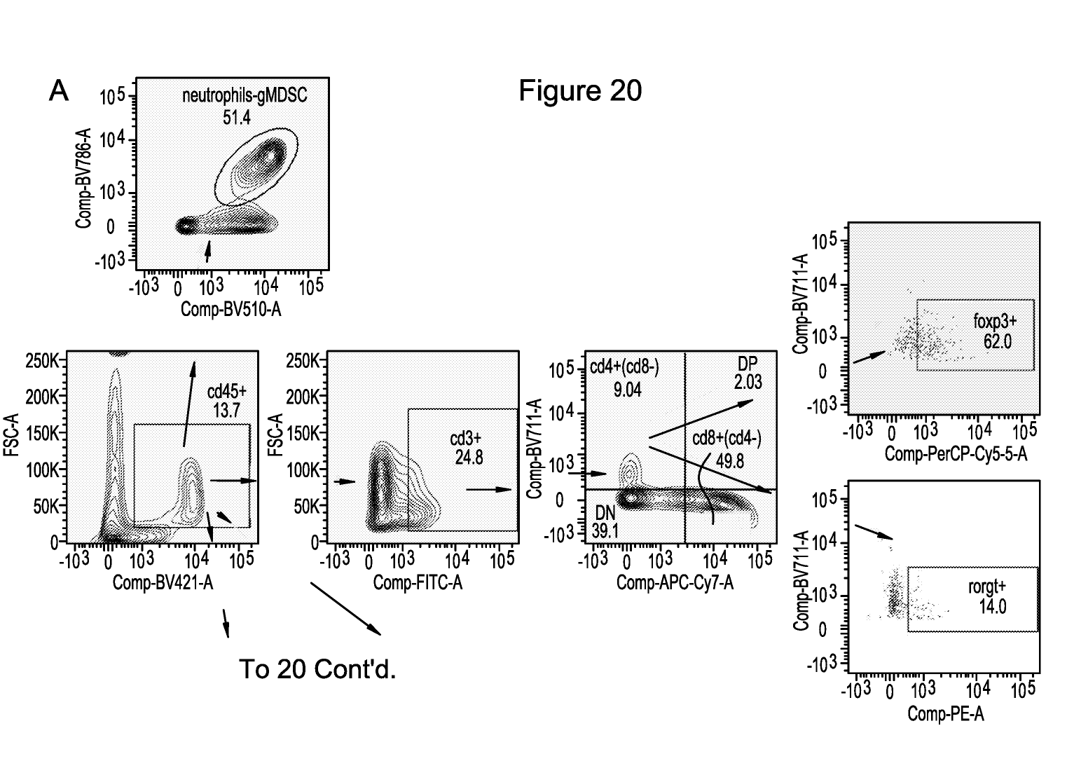

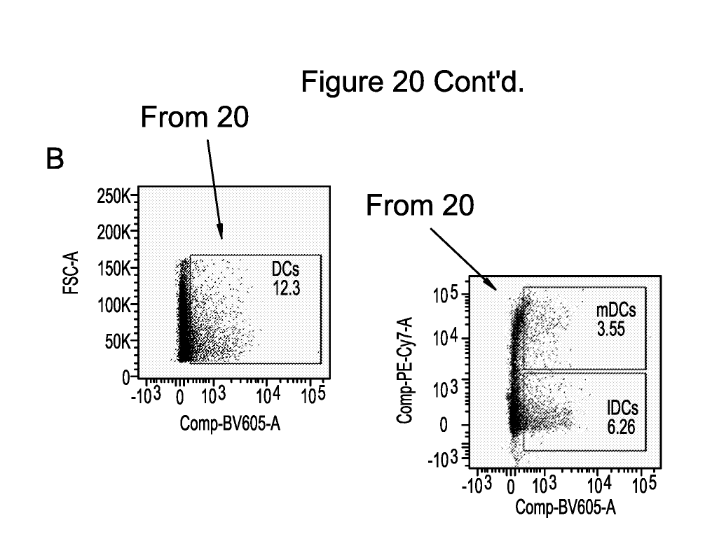

Frequency of lymphoid and myeloid immune cell subsets was determined by multicolor flow cytometry according to the gating schematic illustrated in FIG. 19 for spleen and FIG. 20 for tumor tissue. Full Minus One (FMO) controls were used for gating to identify cells that were positive vs. negative for any particular marker in the panel.

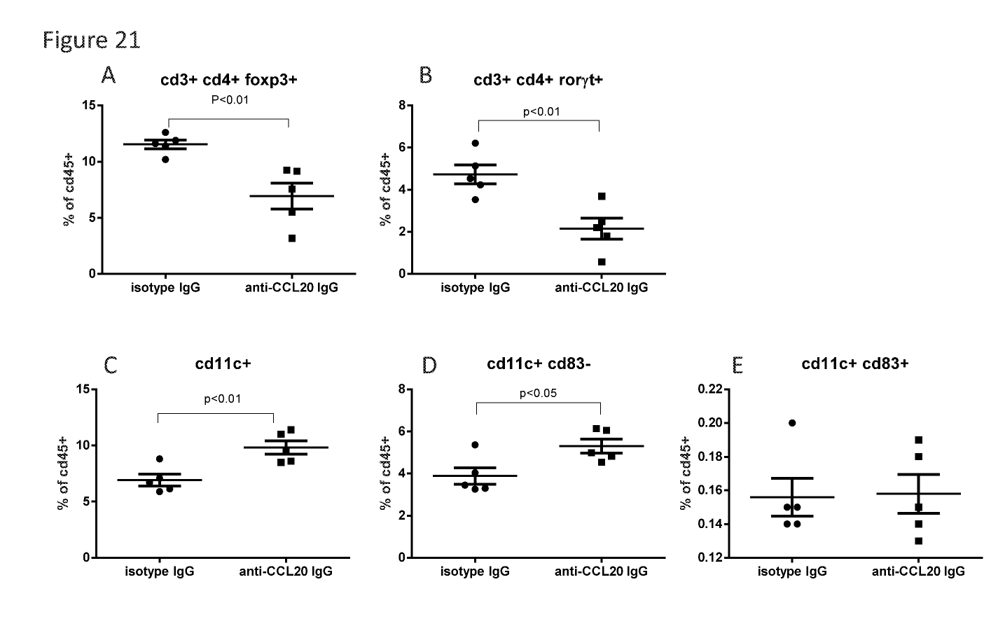

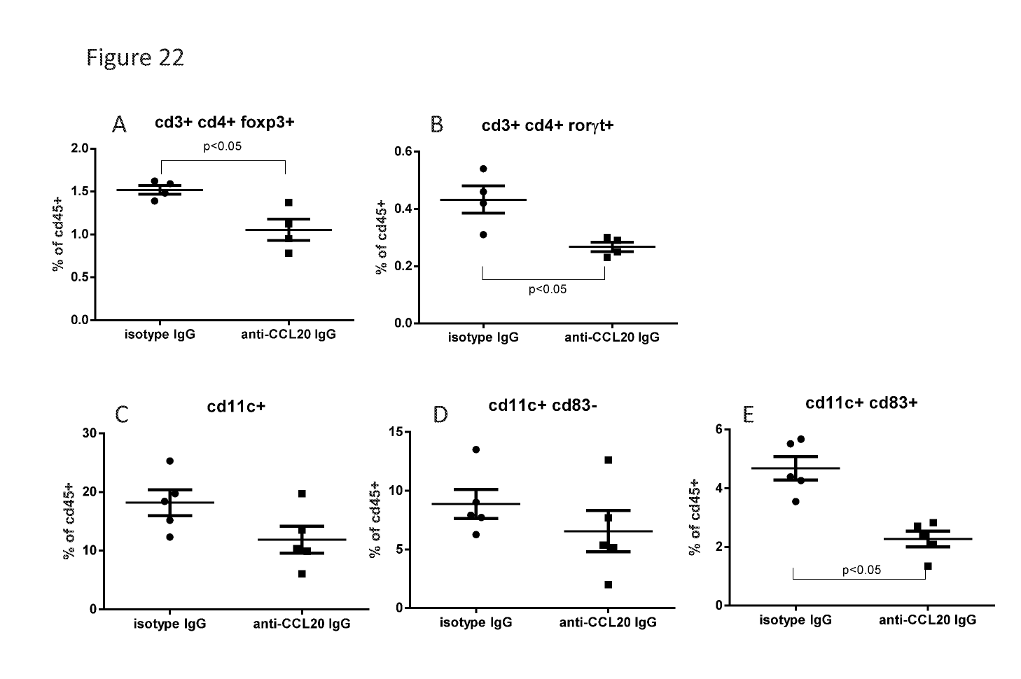

Data indicates that in-vivo treatment with an anti-CCL20 IgG significantly affects the frequency of peripheral and tumor-infiltrating immune cells. In the spleen, CCL20 inhibition caused a significant reduction in foxp3+ T_{regs} (FIG. 21A) and rorγt+ Th₁₇ cells (FIG. 21B), whereby the frequency of both populations decreased approximately 2-fold compared with isotype control treated animals (11.5±0.4% vs. 6.5±1.2% T_{regs} and 4.7±0.4% vs. 2.2±0.5% Th₁₇ in isotype IgG vs. anti-CCL20 IgG treated animals, respectively). This same effect was also captured in the tumor, were the frequency of T_{regs} reduced from 1.5±0.05% following isotype IgG treatment to 1.0±0.1% in response to anti-CCL20 IgG treatment (FIG. 22A), and the frequency of Th₁₇ cells also reduced from 0.4±0.05% in isotype IgG treated tumors to 0.3±0.02% after CCL20 IgG treatment (FIG. 22B). This result suggests that blocking CCL20 in vivo leads to both peripheral and tumor-specific reduction of 2 cell types known to promote immunosuppression in the tumor environment, namely T_{reg} and Th₁₇, suggesting that therapies inhibiting CCL20 can help relieve immunosuppression as part of an anti-cancer therapeutic strategy.

In addition to the effect CCL20 exerts on T_{reg} and Th₁₇ populations, CCL20 neutralization also significantly affected dendritic cells. In the tumor environment, dendritic cell frequency decreased from 18.2±2.2% in isotype IgG treated mice to 11.9±2.3% following CCL20 IgG treatment (FIG. 22C, not significant). This decrease was mostly attributed to the significant reduction in mature cd83+ dendritic cells (FIG. 22E), but not immature cd83-dendritic cells (FIG. 22D). Contrary to this, in the spleen, CCL20 neutralization caused a significant increase in dendritic cells, from 6.9±0.5% in isotype IgG treated mice to 9.8±0.6% in CCL20 IgG treated mice (FIG. 21C). The dendritic cell increase was predominantly to an increase in cd83− immature dendritic cells (FIG. 21D), not cd83+ mature dendritic cells (FIG. 21E). This result indicates that neutralizing CCL20 in vivo inhibits maturation of dendritic cells and consequently infiltration of this cell type into the tumor.

No changes were found in the remaining immune cell populations profiled, namely cd8+ T cells and neutrophils/granulocytic myeloid derived suppressive cells (gMDSC) in either the spleen or tumors of mice treated with anti-CCL20 compared with isotype control treated mice (data not shown).

### DISCUSSION

The data in the above Examples demonstrates a previously unidentified and unexpected function for the CCL20-CCR6 axis aside from its known role in immune cell recruitment. In particular, the studies presented herein demonstrate that with regard to cancer, CCL20 has a specific role in CSC function, representing a previously unreported finding. In several of the illustrative Examples, the results show that in models of breast, colorectal, and pancreatic cancer, CCL20 is specifically overexpressed by CSCs when compared to non-CSC tumor cells, at both the transcript and protein level. The examples also confirm a functional role for the CCL20 protein in CSC activity, as exogenous administration of CCL20 to cell cultures provide a direct increase in the percentage of CSCs as determined by surface marker analysis. CCL20 also increases CSC self-renewal and proliferative function as seen by its effect in directly increasing the stemness gene activation and sphere growth, respectively. In vitro, CCL20 exerts activity on CSCs at physiologically relevant concentrations that are not only similar to the levels secreted into culture medium in our studies, but also similar to the circulating levels detected in colorectal cancer patients with poor prognosis (Iwata et al., 2013).

Although this activity of CCL20 on CSCs was an unexpected finding, other chemokines/cytokines have also been shown to have a direct effect on CSC activity. Interleukin-6 (IL6) and Interleukin-8 (IL8) have both been shown to provide direct effects on breast CSCs (Ginestier et al., 2010; Iliopoulos et al., 2011). Similarly, modulation of CXC-motif ligand 12 (CXCL12) has been shown to modulate glioblastoma CSCs (Wurth et al., 2014), while activity of CCL3 was placed in the context of CML leukemia-initiating cells (Baba et al., 2013). Thus, together with this finding, a growing body of evidence is pointing towards a role for mediators outside of conventional self-renewal pathways such as Notch-, Wnt-, and Hedgehog-signaling to regulate function of CSCs. Given the unique relationship between CCL20 and the receptor CCR6, we hypothesized that CCL20 must exert its activity on CSCs by signaling to its receptor CCR6. By knocking out CCR6, we confirmed that the receptor was indeed required for CCL20's activity in promoting functional CSC activity. But furthermore, when we separated cancer cell lines into fractions that were either positive or negative for the receptor, we found that only the receptor-positive cells showed activation of stemness genes following CCL20 stimulation. Lastly, the observation that CSCs appear to mostly co-localize with a CCL20⁺±/CCR6⁺ cell surface phenotype, suggests that the activity of CCL20 on CSCs could be the result of an autocrine loop, although further studies would be needed to confirm this hypothesis. Altogether, this evidence supports the conclusion that CCL20 exerts its activity on CSCs by signaling through its receptor CCR6 on the tumor cells, and that thus the CCL20-CCR6 axis as a whole can be implemented in CSC activity in breast, pancreatic, and colorectal cancer.

A recent publication demonstrated that APC^{min} mice showed reduced intestinal tumorigenesis when CCR6 was knocked out (Nandi et al., 2014). This result implicated the CCL20-CCR6 axis in tumor initiation in the intestine, again strengthening the role for this axis in the events underlying tumor development and progression. Given our finding that this CCL20-CCR6 axis is relevant to the CSC phenotype, we anticipate that therapeutic intervention of this axis could help eradicate CSCs. In fact, we find that treatment with a neutralizing monoclonal antibody against CCL20 significantly decreases the CSC frequency and activity in models of all three tumor types tested. While we see that CCL20 neutralization directly reduces the percentage of surface phenotype positive CSCs in pancreatic cancer models, we also find that CCL20 neutralization significantly inhibits sphere growth in breast, pancreatic, and colorectal cancer models. These results confirm that we can inhibit not just CSC numbers but, more importantly, CSC function.

In vivo, CSC function can be measured by their potential for tumorigenesis. We found that treatment of CSC-enriched pancreatic cancer spheres with a CCL20 neutralizing monoclonal antibody dramatically delays the onset of tumor formation in mouse models. Moreover, tumors that do form from CSCs treated with anti-CCL20 IgG show a significant reduction in tumor growth compared to isotype control treated tumors. This finding confirms that the role of CCL20 in driving CSC function, identified in the above examples, translates to an in vivo impact on tumorigenesis and rate of tumor growth.

Since the CCL20-CCR6 axis provides a baseline function in immune cell recruitment, we hypothesize that CSC secreted CCL20 could potentially influence tumor-infiltrating lymphocyte recruitment in addition to its direct effect on tumor cells. Chen et al. showed evidence that CCL20 levels directly correlated with regulatory T-cell levels in tumor biopsies of hepatocellular carcinoma patients, and that CCL20 significantly promoted regulatory T-cell migration in this indication (Chen et al., 2011). Given the association of regulatory T-cells with poor prognosis in many tumor types (Want et al. 2012), one could envision a mechanism whereby CCL20 promotes tumorigenicity via a one-two punch, by both regulating the CSC fraction, and by stimulating recruitment of immune-suppressive regulatory T-cells to the tumor environment.

When tested in vivo using immune-competent tumor-bearing mice, we confirmed indeed that CCL20 neutralization inhibits infiltration of T_{regs} into the tumor. However, CCL20 neutralization also inhibited Th₁₇ infiltration into the tumor, a finding that is expected since Th₁₇ cells are characterized as CCR6+. Interestingly, we discovered that CCL20 inhibition not only affects the frequency of these two immune cell types in the tumor, but it also reduces the frequency of T_{regs} and Th₁₇ cells in spleen, suggesting a peripheral response on inhibiting these two cell types beyond mere tumor infiltration. Contrary to this, we also identified that CCL20 neutralization reduced the frequency of mature dendritic cells in the tumor, however, this finding was countered by the presence of increased immature dendritic cells in the spleen. This result suggests that CCL20 also plays a role in maturation and then invasion of dendritic cells into the tumor environment.

Given the association of T_{regs} and Th₁₇ cells with poor prognosis and immunosuppression in many tumor types, and the finding that CCL20 significantly reduces the frequency of these two cell types in the tumor and spleen, our overall findings propose that CCL20 plays a dual role in the tumor environment: 1) by driving activity and tumorigenic ability of CSCs, and 2) by promoting an immunosuppressive phenotype.

An abundance of pre-clinical and clinical evidence points to a critical function for CSCs in cancer treatment, thus treatments targeting this population of tumor cells can be of high value. The work described herein points not only to a novel and important role for the CCL20-CCR6 axis in CSC maintenance, but also to a critical role for CCL20 in supporting an immunosuppressive phenotype. This finding supports the hypothesis that CCL20 acts in a tumor-promoting manner on two fronts, by promoting CSC function and hindering the anti-tumor immune response. Our data indicates that neutralization of CCL20 can be a useful strategy to simultaneously inhibit CSC function and relieve the immunosuppressive phenotype. Overall we hypothesize that, by this dual mechanism, inhibition of CSCs by blocking CCL20 could allow for unique and effective therapies to treat cancer and improve patient lives.

### INCORPORATION BY REFERENCE

All publications and patents mentioned herein are hereby incorporated by reference in their entirety as if each individual publication or patent was specifically and individually indicated to be incorporated by reference.

While specific aspects of the disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### REFERENCES

Al-Hajj, M., Wicha, M. S., Benito-Hernandez, A., Morrison, S. J., and Clarke, M. F. (2003). Prospective identification of tumorigenic breast cancer cells. Proceedings of the National Academy of Sciences of the United States of America 100, 3983-3988.
Baba, M., Imai, T., Nishimura, M., Kakizaki, M., Takagi, S., Hieshima, K., Nomiyama, H., and Yoshie, O. (1997). Identification of CCR6, the specific receptor for a novel lymphocyte-directed CC chemokine LARC. The Journal of biological chemistry 272, 14893-14898.
Baba, T., Naka, K., Morishita, S., Komatsu, N., Hirao, A., and Mukaida, N. (2013). MIP-1alpha/CCL3-mediated maintenance of leukemia-initiating cells in the initiation process of chronic myeloid leukemia. The Journal of experimental medicine 210, 2661-2673.
Barker, N., Ridgway, R. A., van Es, J. H., van de Wetering, M., Begthel, H., van den Born, M., Danenberg, E., Clarke, A. R., Sansom, O. J., and Clevers, H. (2009). Crypt stem cells as the cells-of-origin of intestinal cancer. Nature 457, 608-611.
Chen, K.-J., Lin, S.-Z., Zhou, L., Xie, H.-Y., Zhou, W.-H., Taki-Eldin, A., and Zheng, S.-S. (2011). Selective recruitment of regulatory T cell through CCR6-CCL20 in hepatocellular carcinoma fosters tumor progression and predicts poor prognosis. PloS one 6, e24671.
Creighton, C. J., Li, X., Landis, M., Dixon, J. M., Neumeister, V. M., Sjolund, A., Rimm, D. L., Wong, H., Rodriguez, A., Herschkowitz, J. I., et al. (2009). Residual breast cancers after conventional therapy display mesenchymal as well as tumor-initiating features. Proceedings of the National Academy of Sciences 106, 13820-13825.
Gemei, M., Mirabelli, P., Di Noto, R., Corbo, C., Iaccarino, A., Zamboli, A., Troncone, G., Galizia, G., Lieto, E., Del Vecchio, L., et al. (2013). CD66c is a novel marker for colorectal cancer stem cell isolation, and its silencing halts tumor growth in vivo. Cancer 119, 729-738.
Ghadjar, P., Rubie, C., Aebersold, D. M., and Keilholz, U. (2009). The chemokine CCL20 and its receptor CCR6 in human malignancy with focus on colorectal cancer. International journal of cancer Journal international du cancer 125, 741-745.
Ginestier, C., Liu, S., Diebel, M. E., Korkaya, H., Luo, M., Brown, M., Wicinski, J., Cabaud, O., Charafe-Jauffret, E., Birnbaum, D., et al. (2010). CXCR1 blockade selectively targets human breast cancer stem cells in vitro and in xenografts. The Journal of clinical investigation 120, 485-497.
Hermann, P. C., Huber, S. L., Herrler, T., Aicher, A., Ellwart, J. W., Guba, M., Bruns, C. J., and Heeschen, C. (2007). Distinct populations of cancer stem cells determine tumor growth and metastatic activity in human pancreatic cancer. Cell stem cell 1, 313-323.
Hirsch, D., Barker, N., McNeil, N., Hu, Y., Camps, J., McKinnon, K., Clevers, H., Ried, T., and Gaiser, T. (2014). LGR5 positivity defines stem-like cells in colorectal cancer. Carcinogenesis 35, 849-858.
Iliopoulos, D., Hirsch, H. A., Wang, G., and Struhl, K. (2011). Inducible formation of breast cancer stem cells and their dynamic equilibrium with non-stem cancer cells via IL6 secretion. Proceedings of the National Academy of Sciences of the United States of America 108, 1397-1402.
Iwata, T., Tanaka, K., Inoue, Y., Toiyama, Y., Hiro, J., Fujikawa, H., Okugawa, Y., Uchida, K., Mohri, Y., and Kusunoki, M. (2013). Macrophage inflammatory protein-3 alpha (MIP-3a) is a novel serum prognostic marker in patients with colorectal cancer. Journal of surgical oncology 107, 160-166.
Kleeff, J., Kusama, T., Rossi, D. L., Ishiwata, T., Maruyama, H., Friess, H., Buchler, M. W., Zlotnik, A., and Korc, M. (1999). Detection and localization of Mip-3alpha/LARC/Exodus, a macrophage proinflammatory chemokine, and its CCR6 receptor in human pancreatic cancer. International journal of cancer Journal international du cancer 81, 650-657.
Lapidot, T., Sirard, C., Vormoor, J., Murdoch, B., Hoang, T., Caceres-Cortes, J., Minden, M., Paterson, B., Caligiuri, M. A., and Dick, J. E. (1994). A cell initiating human acute myeloid leukaemia after transplantation into SCID mice. Nature 367, 645-648.
Li, C., Heidt, D. G., Dalerba, P., Burant, C. F., Zhang, L., Adsay, V., Wicha, M., Clarke, M. F., and Simeone, D. M. (2007). Identification of pancreatic cancer stem cells. Cancer research 67, 1030-1037.
Liu, J., Zhang, N., Li, Q., Zhang, W., Ke, F., Leng, Q., Wang, H., Chen, J., and Wang, H. Tumor-associated macrophages recruit CCR6+ regulatory T cells and promote the development of colorectal cancer via enhancing CCL20 production in mice. PLoS One 2011 Apr. 29; 6(4):e19495.
Marsigliante, S., Vetrugno, C., and Muscella, A. (2013). CCL20 induces migration and proliferation on breast epithelial cells. Journal of cellular physiology 228, 1873-1883.
Nandi, B., Pai, C., Huang, Q., Prabhala, R. H., Munshi, N. C., and Gold, J. S. (2014). CCR6, the sole receptor for the chemokine CCL20, promotes spontaneous intestinal tumorigenesis. PloS one 9, e97566.
Schutyser, E., Struyf, S., and Van Damme, J. (2003). The CC chemokine CCL20 and its receptor CCR6. Cytokine & growth factor reviews 14, 409-426.
Tehranchi, R., Woll, P. S., Anderson, K., Buza-Vidas, N., Mizukami, T., Mead, A. J., Astrand-Grundström, I., Strömbeck, B., Horvat, A., Ferry, H., et al. (2010). Persistent Malignant Stem Cells in del(5q) Myelodysplasia in Remission. New England Journal of Medicine 363, 1025-1037.
van Vlerken, L. E., Kiefer, C. M., Morehouse, C., Li, Y., Groves, C., Wilson, S. D., Yao, Y., Hollingsworth, R. E., and Hurt, E. M. (2013). EZH2 is required for breast and pancreatic cancer stem cell maintenance and can be used as a functional cancer stem cell reporter. Stem cells translational medicine 2, 43-52.
Voo, K S, Bover, L, Harline, M L, Vien, L T, Facchinetti V, Arima, K, Kwak, L W, and Liu, Y J. Antibodies targeting human OX40 expand effector T cells and block inducible and natural regulatory T cell function. J. Immunol. 2013 Oct. 1; 191(7): 3641-3650.
Wang, C., Xie, J., Guo, J., Manning, H. C., Gore, J. C., and Guo, N. (2012). Evaluation of CD44 and CD133 as cancer stem cell markers for colorectal cancer. Oncology reports 28, 1301-1308.
Wang, Y., Ma, Y., Fang, Y., Wu, S., Liu, L., Fu, D., and Shen, X. (2012). Regulatory T cell: a protection for tumour cells. Journal of Cellular and Molecular Medicine 16, 425-436.
Wurth, R., Bajetto, A., Harrison, J. K., Barbieri, F., and Florio, T. (2014). CXCL12 modulation of CXCR4 and CXCR7 activity in human glioblastoma stem-like cells and regulation of the tumor microenvironment. Frontiers in cellular neuroscience 8, 144.
Yu, Q., Lou, X. M., and He, Y. Preferential Recruitment of Th17 Cells to Cervical Cancer via CCR6-CCL20 Pathway. PLoS One 2015 Mar. 13; 10(3):e0120855.
Zhang, C. Y., Qi, Y., Li, X. N., Yang, Y., Liu, D. L., Zhao, J., Zhu, D. Y., Wu, K., Zhou, X. D., and Zhao, S. The role of CCL20/CCR6 axis in recruiting Treg cells to tumor sites of NSCLC patients. Biomed Pharmacother 2015 February; 69:242-8.

1. A method for inhibiting proliferation of a cancer stem cell (CSC) comprising contacting the CSC with an inhibitor of CCL20, an inhibitor of CCR6, or a combination thereof, in an amount effective to inhibit proliferation and/or reduce survival of the CSC.

2. The method of claim 1 wherein the CSC expresses CCL20.

3. The method of claim 1 wherein the CSC expresses CCR6.

4. The method of claim 1 wherein the CSC is a sphere-forming CSC.

5. (canceled)

6. (canceled)

7. (canceled)

8. A method for treating a therapeutically-resistant cancer in a subject who has previously received a therapy and who is in need of treatment, comprising administering to the subject an inhibitor of CCL20, an inhibitor of CCR6, or a combination thereof, in an amount effective to treat the therapeutically-resistant cancer.

9. The method of claim 8 wherein the CSCs express CCL20.

10. The method of claim 8 wherein the CSCs express CCR6.

11. The method of claim 8 wherein the CSCs comprise sphere-forming CSCs.

12. (canceled)

13. (canceled)

14. (canceled)

15. A method for treating a cancer that comprises cancer stem cells (CSCs), the method comprising administering to a subject in need of treatment an inhibitor of CCL20, an inhibitor of CCR6, or a combination thereof, in an amount effective to treat the cancer.

16. The method of claim 15 wherein the CSCs express CCL20.

17. The method of claim 15 wherein the CSCs express CCR6.

18. The method of claim 15 wherein the CSCs comprise sphere-forming CSCs.

19. (canceled)

20. (canceled)

21. (canceled)

22. A method for treating cancer in a subject who has recurring or relapsed cancer comprising administering to a subject an inhibitor of CCL20, an inhibitor of CCR6, or a combination thereof, in an amount effective to treat the cancer.

23. The method of claim 22 wherein the CSCs express CCL20.

24. The method of claim 22 wherein the CSCs express CCR6.

25. The method of claim 22 wherein the CSCs comprise sphere-forming CSCs.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A method of determining the presence of a cancer stem cell (CSC) in a sample comprising cancer cells, the method comprising:
contacting the sample with an agent that binds to a CCL20 nucleic acid sequence or a CCL20 amino acid sequence;
detecting the presence or absence of binding between the agent and the CCL20 nucleic acid sequence or the CCL20 amino acid sequence; and
identifying the presence of the CSC in the sample upon detection of binding between the agent and the CCL20 nucleic acid sequence or the CCL20 amino acid sequence.

54. (canceled)

55. (canceled)

56. (canceled)

57. (canceled)

58. (canceled)

59. (canceled)

60. (canceled)

61. (canceled)

62. (canceled)

63. (canceled)

64. A method for reducing or inhibiting tumor growth comprising contacting the tumor with an inhibitor of CCL20, an inhibitor of CCR6, or a combination thereof, in an amount effective to reduce or inhibit tumor growth.

65. The method of claim 64 wherein the tumor expresses CCL20.

66. The method of claim 64 wherein the tumor expresses CCR6.

67. The method of claim 64 wherein the tumor comprises sphere-forming CSCs.

68. (canceled)

69. (canceled)

70. A method for reducing the size of a tumor comprising contacting the tumor with an inhibitor of CCL20, an inhibitor of CCR6, or a combination thereof, in an amount effective to reduce tumor size.

71. The method of claim 70 wherein the tumor expresses CCL20.

72. The method of claim 70 wherein the tumor expresses CCR6.

73. The method of claim 70 wherein the tumor comprises sphere-forming CSCs.

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)


SEQUENCE LISTING (8 sequences)

SEQ ID NO: 1 - DNA, 3156 bp, Homo sapiens
gaaggtccccaggactctgtggtcatcagtaagagagggcccacgtgtatatgctggtgaacagaaatgtcaaccttttcaaagtctgacatttaagagaaaaaactgtggctgttggtttgtggaacagacagctccttctttattgagtcacctctactttcctgctaccgctgcctgtgagctgaaggggctgaaccatacactcctttttctacaaccagcttgcattttttctgcccacaatgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccaccggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtgatagaaagctgagtctccctaaggcatgtgtgaaacatactcatagatgttatgcaaaaaaaagtctatggccaggtatgcatggaaaatgtgggaattaagcaaaatcaagcaagcctctctcctgcgggacttaacgtgctcatgggctgtgtgatctcttcagggtggggtggtctctgataggtagcattttccagcactttgcaaggaatgttttgtagctctagggtatatatccgcctggcatttcacaaaacagcctttgggaaatgctgaattaaagtgaattgttgacaaatgtaaacattttcagaaatattcatgaagcggtcacagatcacagtgtcttttggttacagcacaaaatgatggcagtggtttgaaaaactaaaacagaaaaaaaaatggaagccaacacatcactcattttaggcaaatgtttaaacatttttatctatcagaatgtttattgttgctggttataagcagcaggattggccggctagtgtttcctctcatttccctttgatacagtcaacaagcctgaccctgtaaaatggaggtggaaagacaagctcaagtgttcacaacctggaagtgcttcgggaagaaggggacaatggcagaacaggtgttggtgacaattgtcaccaattggataaagcagctcaggttgtagtgggccattaggaaactgtcggtttgctttgatttccctgggagctgttctctgtcgtgagtgtctcttgtctaaacgtccattaagctgagagtgctatgaagacaggatctagaataatcttgctcacagctgtgctctgagtgcctagcggagttccagcaaacaaaatggactcaagagagatttgattaatgaatcgtaatgaagttggggtttattgtacagtttaaaatgttagatgtttttaattttttaaataaatggaatactttttttttttttttaaagaaagcaactttactgagacaatgtagaaagaagttttgttccgtttctttaatgtggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtggctcacacctgtaatcccagcattttgggaagctaagatgggtggatcacttgaggtcaggagtttgagaccagcctggccaacatggcgaaacccctctctactaaaaatacaaaaatttgccaggcgtggtggcgggtgcctgtaatcccagctacttgggaggctgaggcaagagaatcgcttgaacccaggaggcagaggttgcagtgagccgagatcgtgccattgcactccagcctgggtgacaaagcgagactccatctcaaaaaaaaaaaaaaaaaaaaaggaaagaactgtcatgtaaacataccaacatgtttaaacctgacaatggtgttatttgaaactttatattgttcttgtaagctttaactatatctctctttaaaatgcaaaataatgtcttaagattcaaagtctgtatttttaaagcatggctttggctttgcaaaataaaaaatgtgttttgtacatgaa

SEQ ID NO: 2 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 3 - DNA, 3419 bp, Homo sapiens
tgctcccaggagataaccaggtaaaggagtatgaaagtttgggtacaaactcattgctgcaaattgaaaaccatgcaaaggctgtcttcctctggggagttcaatgcctctctttttcttatcactttaccattggttggactttgattccagggatcctacgattactcaataccctacaggatatacatggttaaccatttgcatttgggcaaataggcgttacttttcaataggaagtggcaatccagaacttgcttttgggcaattctagtagctcaccgcttttttcttaatgactgctagaagctgcatcttattgacagatggtcatcacattggtgagctggagtcatcagattgtggggcccggagtgaggctgaagggagtggatcagagcactgcctgagagtcacctctactttcctgctaccgctgcctgtgagctgaaggggctgaaccatacactcctttttctacaaccagcttgcattttttctgcccacaatgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccaccggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtgatagaaagctgagtctccctaaggcatgtgtgaaacatactcatagatgttatgcaaaaaaaagtctatggccaggtatgcatggaaaatgtgggaattaagcaaaatcaagcaagcctctctcctgcgggacttaacgtgctcatgggctgtgtgatctcttcagggtggggtggtctctgataggtagcattttccagcactttgcaaggaatgttttgtagctctagggtatatatccgcctggcatttcacaaaacagcctttgggaaatgctgaattaaagtgaattgttgacaaatgtaaacattttcagaaatattcatgaagcggtcacagatcacagtgtcttttggttacagcacaaaatgatggcagtggtttgaaaaactaaaacagaaaaaaaaatggaagccaacacatcactcattttaggcaaatgtttaaacatttttatctatcagaatgtttattgttgctggttataagcagcaggattggccggctagtgtttcctctcatttccctttgatacagtcaacaagcctgaccctgtaaaatggaggtggaaagacaagctcaagtgttcacaacctggaagtgcttcgggaagaaggggacaatggcagaacaggtgttggtgacaattgtcaccaattggataaagcagctcaggttgtagtgggccattaggaaactgtcggtttgctttgatttccctgggagctgttctctgtcgtgagtgtctcttgtctaaacgtccattaagctgagagtgctatgaagacaggatctagaataatcttgctcacagctgtgctctgagtgcctagcggagttccagcaaacaaaatggactcaagagagatttgattaatgaatcgtaatgaagttggggtttattgtacagtttaaaatgttagatgtttttaattttttaaataaatggaatactttttttttttttttaaagaaagcaactttactgagacaatgtagaaagaagttttgttccgtttctttaatgtggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtggctcacacctgtaatcccagcattttgggaagctaagatgggtggatcacttgaggtcaggagtttgagaccagcctggccaacatggcgaaacccctctctactaaaaatacaaaaatttgccaggcgtggtggcgggtgcctgtaatcccagctacttgggaggctgaggcaagagaatcgcttgaacccaggaggcagaggttgcagtgagccgagatcgtgccattgcactccagcctgggtgacaaagcgagactccatctcaaaaaaaaaaaaaaaaaaaaaggaaagaactgtcatgtaaacataccaacatgtttaaacctgacaatggtgttatttgaaactttatattgttcttgtaagctttaactatatctctctttaaaatgcaaaataatgtcttaagattcaaagtctgtatttttaaagcatggctttggctttgcaaaataaaaaatgtgttttgtacatgaa

SEQ ID NO: 4 - protein, 374 aa, Homo sapiens; identical to SEQ ID NO: 2

SEQ ID NO: 5 - DNA, 851 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 6 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 7 - DNA, 848 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 8 - protein, 95 aa, Homo sapiens
MCCTKSLLLAALMSVLLLHLCGESEASNFDCCLGYTDRILHPKFIVGFTRQLANEGCDINAIIFHTKKKLSVCANPKQTWVKYIVRLLSKKVKNM